(19) United States
(12) Patent Application Publication
(10) Pub. No.: US 2015/0192593 A1
(43) Pub. Date: Jul. 9, 2015

(54) BIOMARKERS FOR DIAGNOSING AND/OR MONITORING TUBERCULOSIS

(71) Applicant: ProteinLogic Limited, Cambridge (GB)

(72) Inventors: Jane Cunningham, Cambridge (GB); Alexander Betz, Cambridge (GB); Oliver Stegle, Cambridge (GB); Ajit Lilvani, Cambridge (GB)

(21) Appl. No.: 14/418,502

(22) PCT Filed: Jul. 31, 2013
(86) PCT No.: PCT/GB2013/052055

§ 371 Date: Jan. 30, 2015

(30) Foreign Application Priority Data
Jul. 31, 2012 (GB) 1213567.9

(51) Int. Cl. G01N 33/68

(52) U.S. Cl. CPC G01N 33/6872; G01N 33/6866; G01N 33/6869; G01N 33/6893; G01N 33/6863; G01N 2333/70596; G01N 2333/57; G01N 2800/52; G01N 2800/26

(57) ABSTRACT

The invention relates to biomarkers for diagnosing and/or monitoring tuberculosisin both immunocompetent and immunocompromised individuals, monitoring the responses of individuals to anti-mycobacterial chemotherapy, monitoring the progression of latent tuberculosis to active tuberculosis, differentiating active tuberculosis from latent tuberculosis, and from other clinical conditions that mimic tuberculosis (TB). The invention also relates to methods for diagnosing, treating and monitoring tuberculosis using said biomarkers. The above pertain in all aspects both to pulmonary and extrapulmonary Mycobacterium.tuberculosis infections, with Mycobacterium.tuberculosis being the causative organism in tuberculosis.

### FIELD OF THE INVENTION

The invention relates to biomarkers for diagnosing and/or monitoring tuberculosis in both immunocompetent and immunocompromised individuals, monitoring the responses of individuals to anti-mycobacterial chemotherapy, monitoring the progression of latent tuberculosis to active tuberculosis, differentiating active tuberculosis from latent tuberculosis, and from other clinical conditions that mimic tuberculosis (TB). The invention also relates to methods for diagnosing, treating and monitoring tuberculosis using said biomarkers. The above pertain in all aspects both to pulmonary and extrapulmonary Mycobacterium tuberculosis infections, with Mycobacterium tuberculosis being the causative organism in tuberculosis.

### BACKGROUND OF THE INVENTION

Mycobacterium tuberculosis is arguably one of the most successful pathologic micro-organisms worldwide, and is the causative agent of the potentially lethal infectious disease tuberculosis. It is also the leading cause of death worldwide from a potentially curable infectious disease, with an estimated two million related deaths annually.

The pathogenesis of TB is complex, with the initial infection occurring as the result of the inhalation of aerosolized infectious Mycobacterium tuberculosis. The immunological response to the bacillary insult dictates whether the infected individual will proceed to develop either localized pulmonary disease, latent disease (LTBI) or disseminated disease as a consequence of haematogenous spread. Those acquiring latent disease remain asymptomatic but retain the potential to evolve into active disease (this occurs in approximately 10% of all cases over a lifetime period).

It is estimated that approximately one third of the world's population is infected with either latent or active tuberculosis. Recent Health Protection Agency (HPA) figures from 2011 report a total of 9,042 cases of active tuberculosis in the UK. This represents a 5.3% increase on the preceding year, and is suggestive of an upward trend in prevalence in the UK. The number of Mycobacterium tuberculosis isolates resistant to antibiotics is also increasing.

Pulmonary TB is the most common clinical presentation of infection with Mycobacterium tuberculosis. Symptoms typically include chronic cough with or without haemoptysis, fever, night sweats, and weight loss. Only individuals with active pulmonary disease remain infectious as they have the ability to aerosolize bacilli. Infection of almost any other organ system may occur with diverse accompanying clinical presentations. Immunocompromised individuals may present atypically.

The pathogen has shown a dramatic resurgence, driven in part by the HIV epidemic in sub-Saharan Africa, as the immunological response of these individuals is compromised. This has a two fold impact. Firstly, individuals are more likely to progress from pre-existing latent disease to active disease and secondly, primary infection is more likely to take the form of active disease with increased infectivity. With increasing globalisation, the detection, prevention and early appropriate treatment of this aerosolised pathogen is becoming an increasingly important public health priority.

Despite extensive research, the current understanding of the immunological response and pathogenesis of Mycobacterium tuberculosis remains incomplete. Furthermore, the existing diagnostics and treatment methods are suboptimal. Tuberculosis is definitively diagnosed by the identification of the causative organism (Mycobacterium tuberculosis) in a clinical specimen. This is achieved by prolonged culture of the organism, or by PCR analysis. Adjuncts to definitive diagnosis include: diagnostic imaging (X-rays or radiological scans), tuberculin skin tests (Mantoux/Heaf tests) and Interferon Gamma Release Assays (IGRAs).

Existing barriers to rapid definitive TB diagnosis include the difficulty in culturing this slow-growing organism in the laboratory, which can take around 3 to 12 weeks, or in obtaining an appropriate sample containing Mycobacterial DNA for PCR. The latter may require invasive sampling in the case of extrapulmonary TB, which is costly and may involve additional risks to the patient.

The National Institute for Health and Clinical Excellence (NICE) are currently evaluating a PCR-based assay (known as an “Xpert MTB/RIF test”) for use in sputum samples to diagnose cases of suspected active pulmonary tuberculosis, and to detect rifampicin resistant mutations, which are a marker for multi-drug resistant tuberculosis. This methodology is, however, dependent on a PCR-positive sample being obtained.

As a consequence of suboptimal diagnostic tools, anti-microbial combination chemotherapy treatment is often commenced empirically on the basis of clinical suspicion in conjunction with the results of adjunctive diagnostic tests. The rationale for this approach is two fold. The patient receives a therapeutic trial of anti-microbial chemotherapy, and in cases of pulmonary tuberculosis, transmission is curtailed through the reduction of the bacillary load in the sputum.

Current diagnostic adjuncts include: radiological imaging, IGRAs and tuberculin skin testing (TST). Imaging provides guidance as to whether typical features are present, whereas IGRAs and TST provide information regarding possible prior exposure to M. tuberculosis by interrogating the immunological response to TB-related antigens. Interpretation of both the IGRA and TST tests is complex and confounded by a number of factors. These include amongst several others: prior exposure (latent disease), prior vaccination with BCG, and immunosupression. IGRA has, however, increasingly become an accepted adjunctive tool in countries with a low prevalence of TB for evaluating the likelihood of tuberculous disease being present. Unfortunately imaging, TST and the IGRA test are unable to definitively diagnose the presence of active disease.

Recent publications have emphasized the potential for utilising combinations of biomarkers as diagnostic tools for tuberculosis. WO 03/075016 describes that levels of soluble proteins detectable in the blood, namely soluble CD antigens (“sCD”) sCD15, sCD23, sCD27 and sCD54, may be altered in patients with tuberculosis. A Medscape Medical News article from the American Thoracic Society (ATS) 2010 International Conference indicated that a combination of IL-15 and MCP-1 accurately categorised 84% of subjects as having active or latent tuberculosis. Chegou (2^{nd} Global Symposium on IGRAs May-June 2009) describes that combinations of analytes are more promising TB diagnostics than individual analytes and suggests measurement of EGF, sCD40L, MIP-1β, VEGF, TGF-α or IL-1α as a rapid test for active TB. Wu et al (2007) J Immunol 178, 3688-3694 indicated that IL-8, FOXP3 and IL-12β offer a means of differentiating between latent Mycobacterium tuberculosis infection and active tuberculosis disease.

As current diagnostic methodologies for TB are suboptimal, so too are the treatment options available for this disease. As a consequence of the intracellular nature of this pathogen, and lack of adequate innovations in the field of TB therapeutics, the basis of TB therapy continues to be combination anti-microbial chemotherapy over a prolonged period of months in the simplest cases. Partial compliance with treatment may though lead to suboptimal therapeutic levels of the antimicrobials, and the micro-evolution of antibiotic resistance mutations. As a result of this, patients may remain infectious for longer durations, and the frequency of transmission is enhanced. The development of these resistance mutations, some of which are unresponsive to all known anti TB medications (multi-resistant), has recently caused heightened concern in India.

There is therefore a significant need to identify more effective and efficient methods for definitively diagnosing both active and latent TB and in particular differential diagnosis of active and latent TB.

### SUMMARY OF THE INVENTION

According to a first aspect of the invention, there is provided the use of sCD170 as a biomarker for diagnosing and/or monitoring tuberculosis.

According to a second aspect of the invention, there is provided the use of IFN-gamma and sCD170 as biomarkers for diagnosing and/or monitoring tuberculosis.

According to a further aspect of the invention, there is provided a method of diagnosing and/or monitoring tuberculosis, comprising detecting and/or quantifying sCD170, optionally in combination with IFN-gamma and/or one or more additional analyte biomarkers as defined herein, in a clinical sample taken from a test subject.

According to a further aspect of the invention, there is provided a method of diagnosing tuberculosis in an individual thereto, comprising:
(a) obtaining a test biological sample from an individual;
(b) quantifying the amount of sCD170, optionally in combination with IFN-gamma and/or one or more additional analyte biomarkers as defined herein;
(c) comparing the amounts of the analyte biomarkers in the test biological sample with the amounts present in one or more control samples, such that a difference in the level of the analyte biomarkers in the test biological sample is indicative of a diagnosis of tuberculosis.

According to a further aspect of the invention, there is provided a method of monitoring the efficacy of anti-microbial therapy in a subject having or suspected of having tuberculosis, comprising detecting and/or quantifying sCD170, optionally in combination with IFN-gamma and/or one or more additional analyte biomarkers as defined herein, in a sample from said subject.

According to a further aspect of the invention, there is provided a method of determining the efficacy of anti-microbial therapy for tuberculosis in an individual subject comprising:
(a) obtaining a biological sample from an individual;
(b) quantifying the amount of sCD170, optionally in combination with IFN-gamma and/or one or more additional analyte biomarkers as defined herein;
(c) comparing the amounts of the analyte biomarkers in the test biological sample with the amounts present in one or more control samples, such that a difference in the level of the analyte biomarkers in the test biological sample is indicative of a diagnosis of tuberculosis.

According to a further aspect of the invention, there is provided a method of treating tuberculosis in an individual in need thereof, wherein said method comprises the following steps:
(a) diagnosing tuberculosis in an individual according to the methods described herein; followed by
(b) administering an anti-tuberculosis medicament to said individual in the event of a positive diagnosis for tuberculosis.

A further aspect of the invention provides ligands, such as naturally occurring or chemically synthesised compounds, capable of specific binding to the analyte biomarker. A ligand according to the invention may comprise a peptide, an antibody or a fragment thereof, or an aptamer or oligonucleotide, capable of specific binding to the analyte biomarker. The antibody can be a monoclonal antibody or a fragment thereof capable of specific binding to the analyte biomarker. A ligand according to the invention may be labelled with a detectable marker, such as a luminescent, fluorescent or radioactive marker; alternatively or additionally a ligand according to the invention may be labelled with an affinity tag, e.g. a biotin, avidin, streptavidin or His (e.g. hexa-His) tag.

A biosensor according to the invention may comprise the analyte biomarker or a structural/shape mimic thereof capable of specific binding to an antibody against the analyte biomarker. Also provided is an array comprising a ligand or mimic as described herein.

Also provided by the invention is the use of one or more ligands as described herein, which may be naturally occurring or chemically synthesised, and is suitably a peptide, antibody or fragment thereof, aptamer or oligonucleotide, or any other natural or artificial chemical entity capable of recognizing the analyte biomarkers, or the use of a biosensor of the invention, or an array of the invention, or a kit of the invention to detect and/or quantify the analyte. In these uses, the detection and/or quantification can be performed on a biological sample such as from the group consisting of whole blood, serum, plasma, tissue fluid, cerebrospinal fluid (CSF), synovial fluid, follicular fluid, seminal fluid, amniotic fluid, milk, urine, pleural fluid, ascites, bronchoalveolar lavage, saliva, sputum, tears, perspiration, lymphatic fluid, aspirate, bone marrow aspirate and mucus, or an extract or purification therefrom, or dilution thereof.

Diagnostic or monitoring kits are provided for performing methods of the invention. Such kits will suitably comprise a ligand according to the invention, for detection and/or quantification of the analyte biomarker, and/or a biosensor, and/or an array as described herein, optionally together with instructions for use of the kit.

A further aspect of the invention is a kit comprising a biosensor capable of detecting and/or quantifying one or more of the analyte biomarkers as defined herein for use in monitoring or diagnosing tuberculosis.

Biomarkers for tuberculosis are essential targets for the discovery of novel targets and drug molecules that reduce or prevent the progression of symptoms associated with the disorder. As the level of the analyte biomarker is indicative of a diagnosis of the disorder and of the likelihood of a drug response, the biomarker is useful for the identification of novel therapeutic compounds in in vitro and/or in vivo assays. The biomarkers outlined in the invention may be employed in methods for screening for compounds that modulate the activity of the analyte.

Thus, in a further aspect of the invention, there is provided the use of a ligand, as described, which can be a peptide, antibody or fragment thereof or aptamer or oligonucleotide according to the invention, or any other natural or artificial chemical entity capable of recognizing the analyte biomarkers; or the use of a biosensor according to the invention, or an array according to the invention; or a kit according to the invention, to identify a substance capable of promoting and/or of suppressing the generation of the biomarker.

Also there is provided a method of identifying a substance capable of promoting or suppressing the generation of the analyte in a subject, comprising administering a test substance to a subject animal and detecting and/or quantifying the level of the analyte biomarker present in a test sample from the subject.

### BRIEF DESCRIPTION OF THE FIGURES

FIG. 1: Scatter plot of expression levels for IFN gamma, TNF alpha, CD 222 and IL-8

FIG. 2: Scatter plot of expression levels for CD5, CD120b, CD50 and CD170.

FIG. 3: Scatter plot for IL-6, IL-10, CD106, CD26, CD56 and CD85j.

FIG. 4: Scatter plot of IFN gamma versus TNF alpha.

FIG. 5: ROC-curve for differentiation of active/latent using the joint model with all considered antigens.

FIG. 6: ROC-curve for differentiation of healthy/active using the joint model with all considered antigens.

FIG. 7: ROC-curve for differentiation of healthy/latent using the joint model with all considered antigens.

FIG. 8: ROC-curve for differentiation of healthy/TB using the joint model with all considered antigens.

FIG. 9: ROC-curve for differentiation of sick/active using the joint model with all considered antigens.

FIG. 10: ROC-curve for differentiation of sick/latent using the joint model with all considered antigens.

FIG. 11: ROC-curve for differentiation of sick/TB using the joint model with all considered antigens.

FIG. 12: ROC-curve for differentiation of active/latent using the joint model with all considered antigens as in FIG. 5 but repeated with a substantially larger patient sample size.

FIG. 13: ROC-curve for differentiation of active/latent using the joint model with all considered antigens as in FIG. 12 but repeated using the subset of 51 active TB samples and 45 latent TB samples with a positive IGRA test.

FIG. 14: Scatter plot of IFN gamma versus sCD170.

### DETAILED DESCRIPTION OF THE INVENTION

According to a first aspect of the invention, there is provided the use of sCD170 as a biomarker for diagnosing and/or monitoring tuberculosis.

References herein to “sCD170” refer to the secreted or soluble or shed form of CD170 (cluster of differentiation 170). CD170 is also known as SIGLEC5 (Sialic acid-binding Ig-like lectin 5) which is a protein encoded by the SIGLEC5 gene in humans.

Very little information is available on sCD170, however, the information provided herein provides the first link between sCD170 and the diagnosis of tuberculosis.

According to a second aspect of the invention, there is provided the use of IFN-gamma and sCD170 as biomarkers for diagnosing and/or monitoring tuberculosis.

In particular, diagnosing and/or monitoring tuberculosis comprises diagnosing the presence of or monitoring the response to therapeutic intervention of tuberculosis.

References herein to “IFN-gamma” includes also “Interferon-gamma” or “IFN-γ” which is a dimerized soluble cytokine that is the only member of the type II class of interferons. The existence of this interferon, which early in its history was known as immune interferon, was recognized in 1970 when tuberculin-sensitized peritoneal cells were challenged with PPD and resulting supernatants were shown to inhibit growth of vesicular stomatitis virus. That report also contained the basic observation underlying the now widely employed interferon gamma release assay used to test for TB. This interferon was later called macrophage-activating factor, a term now used to describe a larger family of proteins to which IFN-γ belongs. In humans, the IFN-γ protein is encoded by the IFNG gene.

Data is presented herein which describes the effectiveness of the combination of both IFN-gamma and sCD170 in representing highly sensitive and specific diagnostic markers indicative for the diagnosis of tuberculosis.

According to a third aspect of the invention which may be mentioned, there is provided the use of IFN-gamma and TNF-alpha as biomarkers for diagnosing and/or monitoring tuberculosis.

References herein to “TNF-alpha” include also “Tumor Necrosis Factor-alpha”, “TNF-α”, “cachexin” or “cachectin” which is a cytokine involved in systemic inflammation and is a member of a group of cytokines that stimulate the acute phase reaction. It is produced chiefly by activated macrophages, although it can be produced by other cell types as well. The primary role of TNF is in the regulation of immune cells. TNF, being an endogenous pyrogen, is able to induce fever, to induce apoptotic cell death, to induce sepsis (through IL-1 & IL-6 production), to induce cachexia, induce inflammation, and to inhibit tumorigenesis and viral replication. Dysregulation of TNF production has been implicated in a variety of human diseases, including Alzheimer's disease, cancer, major depression, and inflammatory bowel disease (IBD). While still controversial, studies of depression and IBD are currently being linked by TNF levels. Tumor necrosis factor-α can be produced ectopically in the setting of malignancy and parallels parathyroid hormone both in causing secondary hypercalcemia and in the cancers with which excessive production is associated.

Data is presented herein which describes the effectiveness of the combination of both IFN-gamma and TNF-alpha in representing highly sensitive and specific diagnostic markers indicative for the diagnosis of tuberculosis.

In one embodiment, the diagnosis comprises the differential diagnosis of any one of: active tuberculosis and latent tuberculosis; active tuberculosis and healthy control(s); latent tuberculosis and healthy control(s); active tuberculosis and sick control(s); and latent tuberculosis and sick control(s). In a further embodiment, the diagnosis comprises the differential diagnosis of active tuberculosis and latent tuberculosis. Data is presented herein which describes the effectiveness of sCD170 alone of the first invention and the combination of either IFN-gamma and sCD170 of the second aspect of the invention or IFN-gamma and TNF-alpha of the third aspect of the invention in representing highly sensitive and specific differential diagnostic markers indicative of the diagnosis of these key discriminations. In particular, the ability of IFN-gamma and sCD170 to differentiate between active and latent tuberculosis.

Although it will be appreciated that either sCD170 and IFN-gamma of the first and second aspects of the invention or IFN-gamma and TNF-alpha of the third aspect of the invention represent the essential analyte biomarkers of each invention, additional biomarkers for diagnosing tuberculosis could also be used in order to improve the statistical significance of the diagnosis of tuberculosis or of the discriminations between active tuberculosis and latent tuberculosis; active tuberculosis and healthy control(s); latent tuberculosis and healthy control(s); active tuberculosis and sick control(s); and latent tuberculosis and sick control(s).

Thus, in one embodiment, the use of the first and second aspects of the invention additionally comprises one or more further analytes selected from: IL-1β, IL-6, IL-8, IL-10, IL-12p70, sCD4, sCD25, sCD26, sCD32b/c, sCD50, sCD56, sCD66a, sCD83, sCD85j, sCD95, sCD106, sCD120b, sCD121b, sCD127, sCD154, sCD222, sCD226, sCDw329 and TNF alpha. According to a further aspect of the invention, there is provided the use of one or more of: IL-1β, IL-6, IL-8, IL-10, IL-12p70, sCD4, sCD25, sCD26, sCD32b/c, sCD50, sCD56, sCD66a, sCD83, sCD85j, sCD95, sCD106, sCD120b, sCD121b, sCD127, sCD154, sCD222, sCD226, sCDw329 and TNF alpha as a biomarker for diagnosing and/or monitoring tuberculosis.

Thus, in one embodiment, the use of the third aspect of the invention additionally comprises one or more further analytes selected from: IL-1β, IL-6, IL-8, IL-10, IL-12p70, sCD4, sCD25, sCD26, sCD32b/c, sCD50, sCD56, sCD66a, sCD83, sCD85j, sCD95, sCD106, sCD120b, sCD121b, sCD127, sCD154, sCD170, sCD222, sCD226 and sCDw329. According to a further aspect of the invention, there is provided the use of one or more of: IL-1β, IL-6, IL-8, IL-10, IL-12p70, sCD4, sCD25, sCD26, sCD32b/c, sCD50, sCD56, sCD66a, sCD83, sCD85j, sCD95, sCD106, sCD120b, sCD121b, sCD127, sCD154, sCD170, sCD222, sCD226 and sCDw329 as a biomarker for diagnosing and/or monitoring tuberculosis.

According to a further aspect of the invention, there is provided the use of sCD66 as a biomarker for diagnosing and/or monitoring tuberculosis.

In a further embodiment, the use of the first and second aspects of the invention additionally comprises one or more further analytes selected from: IL-6, IL-8, IL-10, sCD25, sCD26, sCD50, sCD56, sCD85j, sCD106, sCD120b, sCD222 and TNF alpha. According to a further aspect of the invention, there is provided the use of one or more of: IL-6, IL-8, IL-10, sCD25, sCD26, sCD50, sCD56, sCD85j, sCD106, sCD120b, sCD222 and TNF alpha as a biomarker for diagnosing and/or monitoring tuberculosis.

In a further embodiment, the use of the third aspect of the invention additionally comprises one or more further analytes selected from: IL-6, IL-8, IL-10, sCD25, sCD26, sCD50, sCD56, sCD85j, sCD106, sCD120b, sCD170 and sCD222. According to a further aspect of the invention, there is provided the use of one or more of: IL-6, IL-8, IL-10, sCD25, sCD26, sCD50, sCD56, sCD85j, sCD106, sCD120b, sCD170 and sCD222 as a biomarker for diagnosing and/or monitoring tuberculosis.

In a further embodiment, the use of the first and second aspects of the invention additionally comprises one or more further analytes selected from: IL-8, sCD25, sCD50, sCD120b, sCD222 and TNF alpha. According to a further aspect of the invention, there is provided the use of one or more of: IL-8, sCD25, sCD50, sCD120b, sCD222 and TNF alpha as a biomarker for diagnosing and/or monitoring tuberculosis.

In a further embodiment, the use of the third aspect of the invention additionally comprises one or more further analytes selected from: IL-8, sCD25, sCD50, sCD120b, sCD170 and sCD222. According to a further aspect of the invention, there is provided the use of one or more of: IL-8, sCD25, sCD50, sCD120b, sCD170 and sCD222 as a biomarker for diagnosing and/or monitoring tuberculosis.

In a further embodiment, the use of the first and second aspects of the invention additionally comprises each of the following further analytes: IL-8, sCD25, sCD50, sCD120b, sCD222 and TNF alpha. According to a further aspect of the invention, there is provided the use of one or more of: IL-8, sCD25, sCD50, sCD120b, sCD222 and TNF alpha as a biomarker for diagnosing and/or monitoring tuberculosis.

In a further embodiment, the use of the third aspect of the invention additionally comprises each of the following further analytes: IL-8, sCD25, sCD50, sCD120b, sCD170 and sCD222. According to a further aspect of the invention, there is provided the use of one or more of: IL-8, sCD25, sCD50, sCD120b, sCD170 and sCD222 as a biomarker for diagnosing and/or monitoring tuberculosis.

In a further embodiment, the use of the first and second aspects of the invention additionally comprises each of the following further analytes: IL-6, IL-8, IL-10, sCD25, sCD26, sCD50, sCD56, sCD85j, sCD106, sCD120b, sCD222 and TNF alpha. According to a further aspect of the invention, there is provided the use of one or more of: IL-6, IL-8, IL-10, sCD25, sCD26, sCD50, sCD56, sCD85j, sCD106, sCD120b, sCD222 and TNF alpha as a biomarker for diagnosing and/or monitoring tuberculosis.

In a further embodiment, the use of the third aspect of the invention additionally comprises each of the following further analytes: IL-6, IL-8, IL-10, sCD25, sCD26, sCD50, sCD56, sCD85j, sCD106, sCD120b, sCD170 and sCD222.

According to a further aspect of the invention, there is provided the use of one or more of: IL-6, IL-8, IL-10, sCD25, sCD26, sCD50, sCD56, sCD85j, sCD106, sCD120b, sCD170 and sCD222 as a biomarker for diagnosing and/or monitoring tuberculosis.

It will be appreciated that known biomarkers for tuberculosis could also be used in order to improve the statistical significance to diagnose tuberculosis to make related clinical discriminations between active and latent disease and so on as outlined previously. For example, in one embodiment, the use of the first, second or third aspects of the invention or any of the embodiments mentioned herein additionally comprises one or more analyte biomarkers selected from: sCD15, sCD23, sCD27, sCD54, IL-15, MCP-1, EGF, sCD40L, MIP-1β, VEGF, TGF-α, FOXP3, IL-12β and IL-1α.

The analyte biomarkers of the invention have the potential to provide a number of key advantages over the existing diagnostic tests for tuberculosis. These include rapid diagnosis from serological samples which are relatively easily obtained with no requirement for overnight incubation with RD1 antigens. This is of particular significance in cases of extrapulmonary TB and in paediatric cases where sputum is often swallowed. It also enhances the potential for TB diagnosis in the absence of Category 3 laboratory provisions. The data also supports enhanced diagnostic capability in immunocompromised individuals. Current immunologically based diagnostic adjuncts including the IGRAs, have a low sensitivity and specificity in diagnosing tuberculosis in HIV infected individuals, primarily because HIV-infected TB patients produce quantitatively less gamma interferon in response to TB-specific antigens than HIV-negative TB patients (Tsiouris et al (2006) J Clin Microbiol. 44(8): 2844-2850). By contrast, the analyte biomarkers of the invention may have great utility in the diagnosis of tuberculosis (including extrapulmonary tuberculosis) in immunocompromised individuals by virtue of the high levels of specificity and sensitivity demonstrated by data shown herein. The data also supports more sensitive and specific discriminations between key clinical discriminations namely: active tuberculosis and latent tuberculosis; active tuberculosis and healthy control(s); latent tuberculosis and healthy control(s); active tuberculosis and sick control(s); and latent tuberculosis and sick control(s).

The analyte biomarkers of the invention also provide the potential for greater comprehension of the immunological response to the disease, and therefore the development of targeted immunotherapy both in respect to the development of immunotherapy for active disease, and the development of post-exposure prophylaxis.

References herein to “tuberculosis” include an infectious disease caused by the presence of the pathogen Mycobacterium tuberculosis. References to tuberculosis also include references to active symptomatic tuberculosis infection and latent asymptomatic tuberculosis infection (LTBI). Most instances of tuberculosis infection are primarily restricted to the lungs (i.e. pulmonary tuberculosis), however, approximately one quarter of active tuberculosis infections move from the lungs, causing other kinds of tuberculosis, collectively referred to as extrapulmonary tuberculosis. This occurs more commonly in immunosuppressed persons (i.e. those suffering from HIV or AIDS) and young children.

In the context of the invention, the term ‘CD’ refers to a cell surface leukocyte molecule recognised by a given monoclonal or group of monoclonal antibodies or polyclonal antibodies which specifically ‘cluster’ to the antigen/molecule in question or a polyclonal antibody. Many, if not all of these CD molecules produce soluble forms that are released from the cell surface by alternative splicing, proteolytic cleavage, dissociation or other mechanisms. Thus in the context of the invention, the term sCD (i.e. soluble CD molecule) is synonymous with the term secreted or soluble or shed CD (sCD) and refers to a released form of a leukocyte molecule that is typically found expressed at the cell surface and in which at least a portion of that molecule is recognised by a given monoclonal or group of monoclonal antibodies or polyclonal antibody as herein described. It should be noted however, that the antibody used to recognise the CD molecule may not be a naturally occurring monoclonal or polyclonal antibody. It may be engineered, an artificial construct consisting of an expressed fragment derived from an antibody molecule with intact recognition, or it may be a non-protein molecular recognition agent, or a protein recognition agent, which is not an antibody, or is an antibody hybrid, for example made by introducing antibody binding sites into a different scaffolding.

Advantageously, as defined in WO 03/075016, a soluble form of sCD is generated by various mechanisms including, but not limited to, any of those selected from the group consisting of the following: alternative splicing, proteolytic cleavage and dissociation.

The term “biomarker” refers to a distinctive biological or biologically derived indicator of a process, event, or condition. Analyte biomarkers can be used in methods of diagnosis, e.g. clinical screening, and prognosis assessment and in monitoring the results of therapy, identifying patients most likely to respond to a particular therapeutic treatment, drug screening and development. Biomarkers and uses thereof are valuable for identification of new drug treatments and for discovery of new targets for drug treatment. In particular, the biomarkers of the invention have the potential to effectively monitor the immunological response to anti-TB therapy. For example, it could be easily established which patients have the potential for a shortened course of treatment (currently tuberculosis treatment for sensitive strains ranges from 6 to 12 months).

In view of the fact that the invention is primarily directed to the diagnosis of an infectious disease, drug resistant mutations of tuberculosis are of particular concern. For example, certain strains of tuberculosis exist which are resistant to particular forms of anti-tuberculosis treatment, such as rifampicin resistant tuberculosis. The above mentioned “monitoring” aspects of the invention are therefore of critical importance because non-response to treatment can be an early sign that the tuberculosis may be rifampicin or multi-drug resistant tuberculosis. In this situation, alternative treatment regimes may be employed at a much earlier phase which will allow a greater possibility for the treated individual to survive and decrease transmission.

According to a further aspect of the invention, there is provided a method of diagnosing tuberculosis in an individual thereto comprising
a) obtaining a test biological sample from an individual;
b) quantifying the amount of sCD170, optionally in combination with IFN-gamma and/or one or more additional analyte biomarkers as defined herein in the test biological sample; and
c) comparing the amounts of sCD170, optionally in combination with IFN-gamma and/or one or more additional analyte biomarkers as defined herein, in the test biological sample with the amounts present in one or more control samples, wherein a higher level of sCD170 in the test biological sample compared with the control sample is indicative of a diagnosis of tuberculosis.

According to a further aspect of the invention, there is provided a method of diagnosing tuberculosis in an individual thereto comprising
a) obtaining a test biological sample from an individual;
b) quantifying the amounts of IFN-gamma and TNF-alpha, optionally in combination with one or more additional analyte biomarkers as defined herein in the test biological sample; and
c) comparing the amounts of IFN-gamma and TNF-alpha, optionally in combination with one or more additional analyte biomarkers as defined herein, in the test biological sample with the amounts present in one or more control samples, wherein a higher level of IFN-gamma and TNF-alpha in the test biological sample compared with the control sample is indicative of a diagnosis of tuberculosis.

In one embodiment, the higher level is a >1 fold difference relative to the control sample, such as a fold difference of 1.5, 2.0, 2.5, 3.0, 3.5, 4.0, 4.5, 5.0, 5.5, 6.0, 6.5, 7.0, 7.5, 8.0, 8.5, 9.0, 9.5, 10, 10.5, 11, 11.5, 12, 12.5, 15, 20, 25, 30, 35, 40, 45, 50, 55, 60, 65, 70, 75, 80, 85, 90, 95, 100 or any ranges there between. In one embodiment, the higher level is between 1 and 75 fold difference relative to the control sample, such as between 1.5 and 10, in particular between 1.5 and 5.

In one embodiment, one or more of the biomarkers may be replaced by a molecule, or a measurable fragment of the molecule, found upstream or downstream of the biomarker in a biological pathway.

As used herein, the term “biosensor” means anything capable of detecting the presence of the biomarker. Examples of biosensors are described herein.

Biosensors according to the invention may comprise a ligand or ligands, as described herein, capable of specific binding to the analyte biomarker. Such biosensors are useful in detecting and/or quantifying an analyte of the invention. Diagnostic kits for the diagnosis and monitoring of tuberculosis are described herein. In one embodiment, the kits additionally contain a biosensor capable of detecting and/or quantifying an analyte biomarker.

Monitoring methods of the invention can be used to monitor onset, progression, stabilisation, amelioration, remission and/or response to therapeutic intervention. It will also be appreciated that monitoring may also include monitoring the extent of tuberculosis to detect the severity of the disease. The markers of the invention may provide differentiation between latent tuberculosis and active tuberculosis. For example, the invention finds great utility in assisting diagnostic capability both in terms of latent disease, active disease and establishing those with latent disease that may progress to active disease.

In methods of diagnosing and/or monitoring according to the invention, detecting and/or quantifying the analyte biomarker in a biological sample from a test subject may be performed on two or more occasions. Comparisons may be made between the level of biomarker in samples taken on two or more occasions. Assessment of any change in the level of the analyte biomarker in samples taken on two or more occasions may be performed. Modulation of the analyte biomarker level is useful as an indicator of the state of tuberculosis. A decrease in the level of the analyte biomarker, over time may be indicative of onset or progression, i.e. worsening of the disorder, whereas an increase in the level of the analyte biomarker indicates amelioration or remission of the disorder, or vice versa.

A method of diagnosis or monitoring according to the invention may comprise quantifying the analyte biomarker in a test biological sample from a test subject and comparing the level of the analyte present in said test sample with one or more controls.

The control used in any one of the methods of the invention defined herein may comprise one or more control samples selected from: the level of analyte biomarker found in a healthy control sample from a healthy individual, a healthy analyte biomarker level; or a healthy analyte biomarker range; patients with other respiratory infections; patients with non-TB mycobacterial infections; and patients known to have active or latent TB.

In one embodiment, there is provided a method of diagnosing tuberculosis, which comprises:
(a) quantifying the amount of sCD170, optionally in combination with IFN-gamma and/or one or more additional analyte biomarkers as defined herein, in a test biological sample; and
(b) comparing the amount of said analytes in said test sample with the amount present in one or more control samples.

In an alternative embodiment, there is provided a method of diagnosing tuberculosis, which comprises:
(a) quantifying the amount of IFN-gamma and TNF-alpha, optionally in combination with one or more additional analyte biomarkers as defined herein, in a test biological sample; and
(b) comparing the amount of said analytes in said test sample with the amount present in one or more control samples.

For biomarkers which are increased in patients with tuberculosis, a higher level of the analyte biomarker in the test sample relative to the level in the healthy control is indicative of a diagnosis of tuberculosis; an equivalent or lower level of the analyte biomarker in the test sample relative to the healthy control is indicative of absence of tuberculosis. For biomarkers which are decreased in patients with tuberculosis, a lower level of the analyte biomarker in the test sample relative to the level in the healthy control is indicative of the diagnosis of tuberculosis; an equivalent or lower level of the analyte biomarker in the test sample relative to the healthy control is indicative of absence of tuberculosis. It will also be appreciated that wherein the control sample comprises a sample obtained from a patient with active or latent tuberculosis, a positive diagnosis of active or latent tuberculosis will typically require a substantially similar level of the analyte biomarker with the control sample.

The term “diagnosis” as used herein encompasses identification, confirmation, and/or characterisation of tuberculosis. Methods of monitoring and of diagnosis according to the invention are useful to confirm the existence of tuberculosis; to monitor development of the disorder by assessing onset and progression, or to assess amelioration or regression of the disorder. Methods of monitoring and of diagnosis are also useful in methods for assessment of clinical screening, prognosis, choice of therapy, evaluation of therapeutic benefit, i.e. for drug screening and drug development.

Efficient diagnosis and monitoring methods provide very powerful “patient solutions” with the potential for improved prognosis, by establishing the correct diagnosis, allowing rapid identification of the most appropriate treatment (thus lessening unnecessary exposure to harmful drug side effects).

Also provided is a method of monitoring efficacy of a therapy for tuberculosis in a subject having such a disorder, suspected of having such a disorder, comprising detecting and/or quantifying the analyte biomarker present in a biological sample from said subject. In monitoring methods, test samples may be taken on two or more occasions. The method may further comprise comparing the level of the biomarker(s) present in the test sample with one or more control(s) and/or with one or more previous test sample(s) taken earlier from the same test subject, e.g. prior to commencement of therapy, and/or from the same test subject at an earlier stage of therapy. The method may comprise detecting a change in the level of the biomarker(s) in test samples taken on different occasions.

The invention provides a method for monitoring efficacy of therapy for tuberculosis in a subject, comprising:
(a) quantifying the amount of sCD170, optionally in combination with IFN-gamma and/or one or more additional analyte biomarkers as defined herein; and
(b) comparing the amount of said sCD170, optionally in combination with IFN-gamma and/or one or more additional analyte biomarkers as defined herein, in said test sample with the amount present in one or more control(s) and/or one or more previous test sample(s) taken at an earlier time from the same test subject.

The invention further provides a method for monitoring efficacy of therapy for tuberculosis in a subject, comprising:
(a) quantifying the amount of IFN-gamma and TNF-alpha, optionally in combination with one or more additional analyte biomarkers as defined herein; and
(b) comparing the amount of said IFN-gamma and TNF-alpha, optionally in combination with one or more additional analyte biomarkers as defined herein, in said test sample with the amount present in one or more control(s) and/or one or more previous test sample(s) taken at an earlier time from the same test subject.

For biomarkers which are increased in patients with tuberculosis, a decrease in the level of the analyte biomarker in the test sample relative to the level in a previous test sample taken earlier from the same test subject is indicative of a beneficial effect, e.g. stabilisation or improvement, of said therapy on the disorder. For biomarkers which are decreased in patients with tuberculosis, an increase in the level of the analyte biomarker in the test sample relative to the level in a previous test sample taken earlier from the same test subject is indicative of a beneficial effect, e.g. stabilisation or improvement, of said therapy on the disorder.

Methods for monitoring efficacy of a therapy can be used to monitor the therapeutic effectiveness of existing therapies and new therapies in human subjects and in non-human animals (e.g. in animal models). These monitoring methods can be incorporated into screens for new drug substances and combinations of substances.

Suitably, the time elapsed between taking samples from a subject undergoing diagnosis or monitoring will be 3 days, 5 days, a week, two weeks, a month, 2 months, 3 months, 6 months, 12 months, 18 months or 24 months. Samples may be taken prior to and/or during and/or following treatment. Samples can be taken at intervals over the remaining life, or a part thereof, of a subject.

The term “detecting” as used herein means confirming the presence of the analyte biomarker present in the sample. Quantifying the amount of the biomarker present in a sample may include determining the concentration of the analyte biomarker present in the sample. Detecting and/or quantifying may be performed directly on the sample, or indirectly on an extract therefrom, or on a dilution thereof.

In alternative aspects of the invention, the presence of the analyte biomarker is assessed by detecting and/or quantifying antibody or fragments thereof capable of specific binding to the biomarker that are generated by the subject's body in response to the analyte and thus are present in a biological sample from a subject having tuberculosis.

Detecting and/or quantifying can be performed by any method suitable to identify the presence and/or amount of a specific analyte biomarker in a biological sample from a patient or a purification or extract of a biological sample or a dilution thereof. In methods of the invention, quantifying may be performed by measuring the concentration of the analyte biomarker in the sample or samples. Biological samples that may be tested in a method of the invention include whole blood, serum, plasma, tissue fluid, cerebrospinal fluid (CSF), synovial fluid, follicular fluid, seminal fluid, amniotic fluid, milk, urine, pleural fluid, ascites, bronchoalveolar lavage, saliva, sputum, tears, perspiration, lymphatic fluid, aspirate, bone marrow aspirate and mucus, or an extract or purification therefrom, or dilution thereof. Biological samples also include tissue homogenates, tissue sections and biopsy specimens from a live subject, or taken post-mortem. The samples can be prepared, for example where appropriate diluted or concentrated, and stored in the usual manner. In one embodiment, the biological sample comprises whole blood, serum or plasma. In a further embodiment, the biological sample comprises serum, such as non-activated or unstimulated serum.

Detection and/or quantification of analyte biomarkers may be performed by detection of the analyte biomarker or of a fragment thereof, e.g. a fragment with C-terminal truncation, or with N-terminal truncation. Fragments are suitably greater than 4 amino acids in length, for example 5, 6, 7, 8, 9, 10, 11, 12, 13, 14, 15, 16, 17, 18, 19, or 20 amino acids in length.

The biomarker may be directly detected, e.g. by SELDI or MALDI-TOF. Alternatively, the biomarker may be detected directly or indirectly via interaction with a ligand or ligands such as an antibody or a biomarker-binding fragment thereof, or other peptide, or ligand, e.g. aptamer, or oligonucleotide, capable of specifically binding the biomarker. The ligand may possess a detectable label, such as a luminescent, fluorescent or radioactive label, and/or an affinity tag.

For example, detecting and/or quantifying can be performed by one or more method(s) selected from the group consisting of: SELDI (-TOF), MALDI (-TOF), a 1-D gel-based analysis, a 2-D gel-based analysis, Mass spectrometry (MS), reverse phase (RP) LC, size permeation (gel filtration), ion exchange, affinity, HPLC, UPLC and other LC or LC MS-based techniques. Appropriate LC MS techniques include ICAT® (Applied Biosystems, CA, USA), or iTRAQ® (Applied Biosystems, CA, USA). Liquid chromatography (e.g. high pressure liquid chromatography (HPLC) or low pressure liquid chromatography (LPLC)), thin-layer chromatography, NMR (nuclear magnetic resonance) spectroscopy could also be used.

Methods of diagnosing and/or monitoring according to the invention may comprise analysing a plasma, serum or whole blood sample by a sandwich immunoassay to detect the presence or level of the analyte biomarker. These methods are also suitable for clinical screening, prognosis, monitoring the results of therapy, identifying patients most likely to respond to a particular therapeutic treatment, for drug screening and development, and identification of new targets for drug treatment.

Detecting and/or quantifying the analyte biomarkers may be performed using an immunological method, involving an antibody, or a fragment thereof capable of specific binding to the analyte biomarker. Suitable immunological methods include sandwich immunoassays, such as sandwich ELISA, in which the detection of the analyte biomarkers is performed using two antibodies which recognize different epitopes on a analyte biomarker; radioimmunoassays (RIA), direct, indirect or competitive enzyme linked immunosorbent assays (ELISA), enzyme immunoassays (EIA), Fluorescence immunoassays (FIA), western blotting, immunoprecipitation and any particle-based immunoassay (e.g. using gold, silver, or latex particles, magnetic particles, or Q-dots). Immunological methods may be performed, for example, in microtitre plate or strip format.

Immunological methods in accordance with the invention may be based, for example, on any of the following methods.

Immunoprecipitation is the simplest immunoassay method; this measures the quantity of precipitate, which forms after the reagent antibody has incubated with the sample and reacted with the target antigen present therein to form an insoluble aggregate. Immunoprecipitation reactions may be qualitative or quantitative.

In particle immunoassays, several antibodies are linked to the particle, and the particle is able to bind many antigen molecules simultaneously. This greatly accelerates the speed of the visible reaction. This allows rapid and sensitive detection of the biomarker.

In immunonephelometry, the interaction of an antibody and target antigen on the biomarker results in the formation of immune complexes, which are too small to precipitate. However, these complexes will scatter incident light and this can be measured using a nephelometer. The antigen, i.e. biomarker, concentration can be determined within minutes of the reaction.

Radioimmunoassay (RIA) methods employ radioactive isotopes such as I¹²⁵ to label either the antigen or antibody. The isotope used emits gamma rays, which are usually measured following removal of unbound (free) radiolabel. The major advantages of RIA, compared with other immunoassays, are higher sensitivity, easy signal detection, and well-established, rapid assays. The major disadvantages are the health and safety risks posed by the use of radiation and the time and expense associated with maintaining a licensed radiation safety and disposal program. For this reason, RIA has been largely replaced in routine clinical laboratory practice by enzyme immunoassays.

Enzyme (EIA) immunoassays were developed as an alternative to radioimmunoassays (RIA). These methods use an enzyme to label either the antibody or target antigen. The sensitivity of EIA approaches that for RIA, without the danger posed by radioactive isotopes. One of the most widely used EIA methods for detection is the enzyme-linked immunosorbent assay (ELISA). ELISA methods may use two antibodies one of which is specific for the target antigen and the other of which is coupled to an enzyme, addition of the substrate for the enzyme results in production of a chemiluminescent or fluorescent signal.

Fluorescent immunoassay (FIA) refers to immunoassays which utilize a fluorescent label or an enzyme label which acts on the substrate to form a fluorescent product. Fluorescent measurements are inherently more sensitive than colorimetric (spectrophotometric) measurements. Therefore, FIA methods have greater analytical sensitivity than EIA methods, which employ absorbance (optical density) measurement.

Chemiluminescent immunoassays utilize a chemiluminescent label, which produces light when excited by chemical energy; the emissions are measured using a light detector.

Immunological methods according to the invention can thus be performed using well-known methods. Any direct (e.g., using a sensor chip) or indirect procedure may be used in the detection of analyte biomarkers of the invention.

The Biotin-Avidin or Biotin-Streptavidin systems are generic labelling systems that can be adapted for use in immunological methods of the invention. One binding partner (hapten, antigen, ligand, aptamer, antibody, enzyme etc) is labelled with biotin and the other partner (surface, e.g. well, bead, sensor etc) is labelled with avidin or streptavidin. This is conventional technology for immunoassays, gene probe assays and (bio)sensors, but is an indirect immobilisation route rather than a direct one. For example a biotinylated ligand (e.g. antibody or aptamer) specific for an analyte biomarker of the invention may be immobilised on an avidin or streptavidin surface, the immobilised ligand may then be exposed to a sample containing or suspected of containing the analyte biomarker in order to detect and/or quantify an analyte biomarker of the invention. Detection and/or quantification of the immobilised antigen may then be performed by an immunological method as described herein.

The term “antibody” as used herein includes, but is not limited to: polyclonal, monoclonal, bispecific, humanised or chimeric antibodies, single chain antibodies, fragments (such as FAb, F(Ab′)₂, Fv, disulphide linked Fv, scFv, diabody), fragments produced by a Fab expression library, anti-idiotypic (anti-Id) antibodies and epitope-binding fragments of any of the above. The term “antibody” as used herein also refers to immunoglobulin molecules and immunologically-active portions of immunoglobulin molecules, i.e., molecules that contain an antigen binding site that specifically binds an antigen. The immunoglobulin molecules of the invention can be of any class (e. g., IgG, IgE, IgM, IgD and IgA) or subclass of immunoglobulin molecule.

The identification of key biomarkers specific to a disease is central to integration of diagnostic procedures and therapeutic regimes. Using predictive biomarkers appropriate diagnostic tools such as biosensors can be developed; accordingly, in methods and uses of the invention, detecting and quantifying can be performed using a biosensor, microanalytical system, microengineered system, microseparation system, immunochromatography system or other suitable analytical devices. The biosensor may incorporate an immunological method for detection of the biomarker(s), electrical, thermal, magnetic, optical (e.g. hologram) or acoustic technologies. Using such biosensors, it is possible to detect the target biomarker(s) at the anticipated concentrations found in biological samples.

Thus, according to a further aspect of the invention there is provided an apparatus for diagnosing and/or monitoring tuberculosis which comprises a biosensor, microanalytical, microengineered, microseparation and/or immunochromatography system configured to detect and/or quantify any of the analyte biomarkers defined herein.

Suitably, biosensors for detection of one or more biomarkers of the invention combine biomolecular recognition with appropriate means to convert detection of the presence, or quantitation, of the biomarker in the sample into a signal. Biosensors can be adapted for “alternate site” diagnostic testing, e.g. in the ward, outpatients' department, surgery, home, field and workplace.

Biosensors to detect one or more biomarkers of the invention include acoustic, plasmon resonance, holographic and microengineered sensors. Imprinted recognition elements, thin film transistor technology, magnetic acoustic resonator devices and other novel acousto-electrical systems may be employed in biosensors for detection of the one or more biomarkers of the invention.

Levels of the analyte biomarkers of the second and third aspects of the invention IFN-gamma and TNF-alpha may be measured in accordance with any of the techniques described hereinbefore. In one particular embodiment, the levels of IFN-gamma and TNF-alpha may be measured in accordance with an MSD® assay, such as the Human Pro-inflammatory 7-Plex Assay Ultra-Sensitive Kit (Mesoscale Discovery; Catalogue No. K15008C-1, K15008C-2 or K15008C-4).

This Pro-inflammatory assay detects IFN-gamma or TNF-alpha in a sandwich immunoassay format by providing a plate which has been pre-coated with capture antibody on spatially distinct spots. A sample is then added to the plate along with a solution containing anti-IFN-gamma or anti-TNF-alpha detection antibodies labelled with an electrochemiluminescent compound and incubated for a specified time period. IFN-gamma or TNF-alpha within the sample will bind to capture antibodies immobilised on the working electrode surface and recruitment of the labelled detection antibodies by bound IFN-gamma analytes or TNF-alpha analytes completes the sandwich. An MSD read buffer is then added to provide a chemical environment for electrochemilluminescence and the plate is then loaded into an MSD SECTOR® instrument for analysis. Inside the SECTOR instrument, a voltage applied to the plate electrodes causes the labels bound to the electrode surface to emit light. The instrument measures intensity of emitted light to afford a quantitative measure of IFN-gamma or TNF-alpha present in the sample. It will be appreciated that other cytokines and chemokines (such as IL-1α, IL-1β, IL-6, IL-8, IL-10, IL-12β, IL-15, IL-12p70, MCP-1, EGF, MIP-1β, VEGF, TGF-α and FOXP3) may be quantified in accordance with analogous procedures described above for IFN-gamma and TNF-alpha.

When the analyte biomarker comprises a CD molecule, such as sCD4, sCD15, sCD23, sCD25, sCD26, sCD27, sCD32b/c, sCD40L, sCD50, sCD54, sCD56, sCD66a, sCD83, sCD85j, sCD95, sCD106, sCD120b, sCD121b, sCD127, sCD154, sCD170, sCD222, sCD226 or sCDw329, levels may be measured from samples of serum or plasma or other body fluids using reagents suitable for detecting soluble CDs that include but are not limited to antibodies raised against those CDs. In one embodiment, monoclonal antibodies or engineered antibodies, including phage antibodies raised against the sCD or their membrane bound form are used for their detection. However, non-protein agents may also in principle be used to detect sCDs. Similarly the detecting molecule may contain antibody binding site fragments incorporated into the scaffold of another molecule or an engineered scaffold. Commercially available kits for measuring CD levels include those from Diaclone 1, Bd A Fleming BP 1985 F-25020 Besancon Cedex-France and Medsystems Diagnostics GmbH, Rennweg 95b, A-1030 Vienna Austria.

Suitable techniques for measuring sCDs include but are not limited to immunoassays including ELISA using commercially available kits such as those described above, flow cytometry particularly multiplexed particle flow cytometry as herein described. Those skilled in the art will be aware of other suitable techniques for measuring CD levels in samples from an individual including antibody ‘chip’ array type technologies or chip technologies utilizing non-classical antibody binding site grafted molecules.

In one particular embodiment, the method of measuring sCDs comprises an MSD® assay as defined hereinbefore with the modifications described herein.

Methods involving detection and/or quantification of one or more analyte biomarkers of the invention can be performed on bench-top instruments, or can be incorporated onto disposable, diagnostic or monitoring platforms that can be used in a non-laboratory environment, e.g. in the physician's office or at the patient's bedside. Suitable biosensors for performing methods of the invention include “credit” cards with optical or acoustic readers. Biosensors can be configured to allow the data collected to be electronically transmitted to the physician for interpretation and thus can form the basis for e-neuromedicine.

Any suitable animal may be used as a subject non-human animal, for example a non-human primate, horse, cow, pig, goat, sheep, dog, cat, fish, rodent, e.g. guinea pig, rat or mouse; insect (e.g. Drosophila), amphibian (e.g. Xenopus) or C. elegans.

The test substance can be a known chemical or pharmaceutical substance, such as, but not limited to, an anti-depressive disorder therapeutic; or the test substance can be a novel synthetic or natural chemical entity, or a combination of two or more of the aforesaid substances.

There is provided a method of identifying a substance capable of promoting or suppressing the generation of the analyte biomarker in a subject, comprising exposing a test cell to a test substance and monitoring the level of the analyte biomarker within said test cell, or secreted by said test cell.

The test cell could be prokaryotic, however a eukaryotic cell will suitably be employed in cell-based testing methods. Suitably, the eukaryotic cell is a yeast cell, insect cell, Drosophila cell, amphibian cell (e.g. from Xenopus), C. elegans cell or is a cell of human, non-human primate, equine, bovine, porcine, caprine, ovine, canine, feline, piscine, rodent or murine origin.

In methods for identifying substances of potential therapeutic use, non-human animals or cells can be used that are capable of expressing the analyte.

Screening methods also encompass a method of identifying a ligand capable of binding to the analyte biomarker according to the invention, comprising incubating a test substance in the presence of the analyte biomarker in conditions appropriate for binding, and detecting and/or quantifying binding of the analyte to said test substance.

High-throughput screening technologies based on the biomarker, uses and methods of the invention, e.g. configured in an array format, are suitable to monitor biomarker signatures for the identification of potentially useful therapeutic compounds, e.g. ligands such as natural compounds, synthetic chemical compounds (e.g. from combinatorial libraries), peptides, monoclonal or polyclonal antibodies or fragments thereof, which may be capable of binding the biomarker.

Methods of the invention can be performed in array format, e.g. on a chip, or as a multiwell array. Methods can be adapted into platforms for single tests, or multiple identical or multiple non-identical tests, and can be performed in high throughput format. Methods of the invention may comprise performing one or more additional, different tests to confirm or exclude diagnosis, and/or to further characterise a condition.

The invention further provides a substance, e.g. a ligand, identified or identifiable by an identification or screening method or use of the invention. Such substances may be capable of inhibiting, directly or indirectly, the activity of the analyte biomarker, or of suppressing generation of the analyte biomarker. The term “substances” includes substances that do not directly bind the analyte biomarker and directly modulate a function, but instead indirectly modulate a function of the analyte biomarker. Ligands are also included in the term substances; ligands of the invention (e.g. a natural or synthetic chemical compound, peptide, aptamer, oligonucleotide, antibody or antibody fragment) are capable of binding, suitably specific binding, to the analyte.

The invention further provides a substance according to the invention for use in the treatment of tuberculosis.

Also provided is the use of a substance according to the invention in the treatment of tuberculosis.

Also provided is the use of a substance according to the invention as a medicament.

Yet further provided is the use of a substance according to the invention in the manufacture of a medicament for the treatment of tuberculosis.

According to a further aspect of the invention, there is provided a method of treating tuberculosis in an individual in need thereof, wherein said method comprises the following steps:
(a) diagnosing tuberculosis in an individual according to the method described herein; followed by
(b) administering an anti-tuberculosis medicament to said individual in the event of a positive diagnosis for tuberculosis.

In one embodiment, said anti-tuberculosis medicament is: one or more first line medicaments selected from: ethambutol, isoniazid, pyrazinamide, rifampicin; and/or one or more second line medicaments selected from: aminoglycosides (e.g., amikacin, kanamycin), polypeptides (e.g., capreomycin, viomycin, enviomycin), fluoroquinolones (e.g., ciprofloxacin, levofloxacin, moxifloxacin), thioamides (e.g. ethionamide, prothionamide), cycloserine (closerin) or terizidone; and/or one or more third line medicaments selected from rifabutin, macrolides (e.g., clarithromycin), linezolid, thioacetazone, thioridazine, arginine, vitamin D and R207910.

A kit for diagnosing and/or monitoring tuberculosis is provided. Suitably a kit according to the invention may contain one or more components selected from the group: a ligand specific for the analyte biomarker or a structural/shape mimic of the analyte biomarker, one or more controls, one or more reagents and one or more consumables; optionally together with instructions for use of the kit in accordance with any of the methods defined herein. In one embodiment, the kit differentially diagnoses active and latent tuberculosis.

The identification of biomarkers for tuberculosis permits integration of diagnostic procedures and therapeutic regimes. Currently there are significant delays in determining effective treatment and hitherto it has not been possible to perform rapid assessment of drug response. Traditionally, many tuberculosis therapies have required treatment trials lasting weeks to months for a given therapeutic approach. Detection of an analyte biomarker of the invention can be used to screen subjects prior to their participation in clinical trials. The biomarkers provide the means to indicate therapeutic response, failure to respond, unfavourable side-effect profile, degree of medication compliance and achievement of adequate serum or plasma drug levels. The biomarkers may be used to provide warning of adverse drug response. Biomarkers are useful in development of personalized therapies, as assessment of response can be used to fine-tune dosage, minimise the number of prescribed medications, reduce the delay in attaining effective therapy and avoid adverse drug reactions. Thus by monitoring a biomarker of the invention, patient care can be tailored precisely to match the needs determined by the disorder and the pharmacogenomic profile of the patient, the biomarker can thus be used to titrate the optimal dose, predict a positive therapeutic response and identify those patients at high risk of severe side effects.

Biomarker-based tests provide a first line assessment of ‘new’ patients, and provide objective measures for accurate and rapid diagnosis, in a time frame and with precision, not achievable using the current subjective measures.

Biomarker monitoring methods, biosensors and kits are also vital as patient monitoring tools, to enable the physician to determine whether relapse is due to worsening of the disorder, poor patient compliance or drug resistance. In particular, the invention may also be used to monitor patient compliance with taking a particular drug (agent), and/or undergoing a particular treatment regime.

If pharmacological treatment is assessed to be inadequate, then therapy can be reinstated or increased; a change in therapy can be given if appropriate. As the biomarkers are sensitive to the state of the disorder, they provide an indication of the impact of drug therapy.

The following studies illustrate the invention.

### Example 1

### Effectiveness of IFN-Gamma and TNF-Alpha as TB Biomarkers

The use of IFN-gamma, TNF-alpha and additional CDs were assessed for their effectiveness to discriminate between different sample classes (healthy, latent TB, active TB, sick).
1. The combination of IFN-gamma and TNF-alpha consistently yields better discriminative signals than either of these antigens alone.
2. Additional CDs were identified, which further improve the predictive signatures for some of the prediction tasks.

A study was conducted on n=92 human samples to identify potential markers capable of differentiating between subjects with tuberculosis and control patients without tuberculosis.

The demographics of each patient is summarised in Table 1:

TABLE 1
Demographic and Patient Data for Each Sample Analysed

| | Diag- | | | | | |
|---|---|---|---|---|---|---|
| | nostic | | | | | |
| Cohort | Cate- | History | | | | Co- |
| Group | gory | of BCG? | Site of TB | Age | Sex | Morbidity |
| A | 4C | Yes | N/A | 52 | M | Psoriasis |
| A | 4A | Yes | N/A | 36 | M | None |
| A | 4C | Yes | N/A | 43 | M | Diabetes |
| | | | | | | mellitus, |
| | | | | | | Epilepsy, |
| | | | | | | Hypertension |
| A | 4B | Yes | N/A | 34 | F | None |
| A | 4B | Yes | N/A | 26 | F | None |
| A | 4B | Yes | N/A | 43 | M | None |
| A | 4A | Yes | N/A | 32 | M | None |
| A | 4B | Yes | N/A | 37 | M | None |
| A | 4B | Unknown | N/A | 61 | M | None |
| A | 4B | Yes | N/A | 32 | F | None |
| A | 4B | No | N/A | 26 | M | None |
| A | 4C | Yes | N/A | 25 | M | None |
| A | 4C | No | N/A | 30 | F | None |
| A | 4B | Yes | N/A | 34 | F | None |
| A | 4B | Unknown | N/A | 24 | F | None |
| A | 4B | No | N/A | 48 | M | None |
| A | 4B | No | N/A | 40 | M | None |
| A | 4B | No | N/A | 27 | F | None |
| A | 4B | Yes | N/A | 26 | M | None |
| A | 4B | Unknown | N/A | 62 | F | None |
| A | 4B | No | N/A | 33 | F | None |
| A | 4B | No | N/A | 19 | F | None |
| A | 4B | Yes | N/A | 77 | F | Lymphoma, |
| | | | | | | Breast |
| | | | | | | Cancer, |
| | | | | | | COPD |
| A | 4B | Yes | N/A | 62 | M | Diabetes |
| | | | | | | mellitus, |
| | | | | | | Psoriasis |
| A | 4B | Yes | N/A | 35 | M | None |
| A | 4B | Yes | N/A | 38 | M | None |
| A | 4B | No | N/A | 65 | M | None |
| A | 4B | No | N/A | 32 | F | None |
| A | 4C | Yes | N/A | 34 | F | None |
| A | 4B | Yes | N/A | 46 | M | None |
| A | 4B | Yes | N/A | 30 | F | None |
| A | 4B | No | N/A | 57 | F | Hyperthyroid |
| A | 4C | Yes | N/A | 29 | F | None |
| A | 4C | Yes | N/A | 32 | M | None |
| A | 4B | Yes | N/A | 50 | F | None |
| A | 4C | Yes | N/A | 36 | F | None |
| A | 4B | Yes | N/A | 35 | F | None |
| A | 4B | Yes | N/A | 34 | F | None |
| A | 4B | Yes | N/A | 42 | F | None |
| A | 4B | Yes | N/A | 23 | M | None |
| B | 1 | Unknown | Pulmonary | 47 | M | None |
| B | 1 | Unknown | Pulmonary | 34 | M | None |
| B | 1 | Unknown | Pulmonary | 40 | M | None |
| B | 2 | Yes | Pulmonary | 33 | F | None |
| B | 1 | Yes | Pulmonary | 48 | F | None |
| B | 1 | Yes | Pulmonary | 35 | F | Post |
| | | | | | | traumatic |
| | | | | | | stress |
| | | | | | | disorder |
| B | 1 | Yes | Pulmonary | 26 | M | None |
| B | 1 | Yes | Pulmonary | 20 | M | Epilepsy |
| B | 2 | Yes | Pulmonary | 61 | F | None |
| B | 1 | Unknown | Pulmonary | 35 | M | None |
| B | 1 | Yes | Pulmonary | 31 | M | None |
| B | 1 | Yes | Pulmonary | 22 | M | None |
| B | 1 | Unknown | Pulmonary | 27 | F | None |
| B | 1 | No | Pulmonary | 30 | M | None |
| B | 1 | Yes | Pulmonary | 33 | M | None |
| B | 1 | Yes | Pulmonary | 28 | F | None |
| B | 2 | Unknown | Pulmonary | 32 | M | None |
| | | | and | | | |
| | | | Abdomen | | | |
| B | 1 | Yes | Pulmonary | 50 | F | Asthma |
| B | 2 | Yes | Pulmonary | 42 | M | Diabetes |
| | | | and Bone | | | Mellitus |
| B | 1 | No | Pulmonary | 61 | M | None |
| B | 2 | Yes | Pulmonary + | 38 | F | Retinal |
| | | | Uveitis | | | Vasculitis |
| B | 1 | Yes | Neck | 40 | M | HIV |
| | | | Lymph | | | |
| | | | Node | | | |
| B | 1 | Yes | Pulmonary | 55 | M | COPD |
| B | 2 | Yes | Spine | 41 | F | None |
| B | 2 | Yes | Pulmonary | 20 | F | None |
| B | 1 | Yes | Pulmonary | 21 | M | None |
| | | | (Millary) | | | |
| B | 2 | Yes | Pulmonary | 43 | M | None |
| B | 1 | Yes | Pulmonary | 31 | M | None |
| B | 2 | Yes | Pulmonary | 36 | F | None |
| B | 1 | Yes | Pulmonary | 50 | F | Asthma |
| B | 1 | Yes | Pulmonary | 29 | M | None |
| B | 2 | Yes | Neck | 54 | F | None |
| | | | Lymph | | | |
| | | | Node | | | |
| B | 1 | Yes | Pulmonary | 68 | F | None |
| B | 1 | Yes | Pulmonary | 22 | M | None |
| B | 2 | No | Pulmonary | 32 | M | None |
| B | 1 | Yes | Pulmonary | 28 | F | None |
| B | 2 | Yes | Pulmonary | 29 | F | None |
| C | 4D | Yes | N/A | 34 | F | None |
| C | 4D | Yes | N/A | 30 | M | None |
| C | 4D | Yes | N/A | 38 | F | None |
| C | 4D | Yes | N/A | 23 | M | None |
| C | 4D | Yes | N/A | 35 | M | None |
| C | 4D | Yes | N/A | 23 | F | None |
| C | 4D | Yes | N/A | 33 | M | None |
| C | 4D | Yes | N/A | 25 | F | None |
| C | 4D | Yes | N/A | 28 | F | None |
| C | 4D | Yes | N/A | 31 | M | None |
| C | 4D | Yes | N/A | 24 | F | None |
| C | 4D | Yes | N/A | 22 | F | None |
| C | 4D | Yes | N/A | 23 | F | None |
| C | 4D | Yes | N/A | 23 | F | None |
| C | 4D | Yes | N/A | 23 | F | None |

NB: Cohort A = Latent TB, Cohort B = Active TB, and Cohort C = Controls.
The diagnostic category was calculated in accordance with Table 1 as described in Dosanjh et al (2008) Ann Int Med 148, 325-336.

The analyses were conducted by detecting the amounts of a large number of potential analytes using a Meso Scale Discovery® (MSD) Sector 6000 instrument. For example, the biomarkers of the invention were detected as follows:

### IFN-Gamma, TNF-Alpha, IL-6, IL-8 and IL-10

These markers were quantified using a Human Pro-inflammatory 7-Plex Assay (Meso Scale Discovery Catalogue Numbers K15008C-1, K15008C-2 or K15008C-4) exactly in accordance with the manufacturer's instructions. It will be appreciated that other cytokines and chemokines (such as IL-1α, IL-1β, IL-12β, IL-15, IL-12p70, MCP-1, EGF, MIP-1β, VEGF, TGF-α and FOXP3) may be quantified in accordance with analogous procedures described above for IFN-gamma, TNF-alpha, IL-6, IL-8 and IL-10.

sCD25, sCD26, sCD50, sCD56, sCD85j, sCD106, sCD120b, sCD170 and sCD222

These markers were quantified using a modified Meso Scale Discovery assay by using the following reagents and protocol:

### Reagents Used

Human IGF-IIR (DuoSet DY2447 from R&D systems: Lot 1272152: expiry 25.10.15)

### Capture Antibody

Diluted 1 in 180 in PBS (19 μl in 3.5 ml)
Pipetted 30 μl/well of an MSD standard bind plate
Seal+Incubate at +4 overnight
Wash ×3 with MSD wash buffer
Block for a minimum of 1 hour with 150 μl MSD Blocker A
Wash ×1 with MSD wash buffer

### Standard

Reconstitute with 0.5 ml 1% BSA/PBS=290 ng/ml=290,000 pg/ml
Dilute 1 in 5 in DELFIA Dil II=58,000 pg/ml (50+200)=std 7
Then serially dilute 1 in 2 in DELFIA Dil II (100+100)=29000, 14500, 7250, 3625, 1813, +0 pg/ml
QCs:
   1=pooled plasma 1.2.11
   2=spiked plasma 1.2.11
   3=spiked pool 26.7.10

### Assay

Pipette 40 μl DELFIA Dil II/well+10 μl std/QC/unknown in duplicate
Cover & incubate on a plate shaker 2 hours at Room Temperature
Wash ×3 with MSD wash buffer

### Biotinylated Antibody

Dilute the antibody 1 in 180 in MSD Diluent 100 (19 μl in 3.5 ml)
Add 25 μl/well
Cover and incubate on a plate shaker 1 hour at RT
Wash ×3 with MSD wash buffer

### Strepavidin-SulphoTAG

Dilute 1 in 1000 in MSD Diluent 100 (3 μl in 3 ml)
Add 25 μl/well
Cover and incubate on a plate shaker for 30 minutes at Room Temperature
Wash ×3
150 μl read buffer+read on MSD Sector 6000® reader
Results are calculated using MSD Workbench Software® package

It will be appreciated that other soluble cluster of differentiation (sCD) molecules (such as sCD4, sCD15, sCD23, sCD27, sCD32b/c, sCD40L, sCD54, sCD66a, sCD83, sCD95, sCD121b, sCD127, sCD154, sCD226 or sCDw329) may be quantified in accordance with analogous procedures described above for sCD25, sCD26, sCD50, sCD56, sCD85j, sCD106, sCD120b, sCD170 and sCD222.

### Results

### RAW Data Processing and Normalization

Antigen levels were variance stabilized to decouple the relationship between mean expression level and technical replicate variance. This transformation approximately corresponds to log transforming the data with an additional offset and scale and is standard for most microarray analysis.

### Cross Validation Analyses to Assess Predictive Performance

We assessed the predictive ability of alternative combinations of antigens for the task of discriminating different sample classes. All experiments were conducted using 10-fold cross validation. For each fold, the fraction of 9/10 of the data were used to train a non-linear support vector machine (SVM) classifier to predict the sample label of the remaining 1/10 of the samples. The prediction accuracy was assessed and reported using the area under the curve receiver operating characteristics (AUC). A perfect predictor would yield an AUC of 1.0, whereas a random predictor corresponds to an AUC of 0.5 when the sample set is balanced. A consistently failing predictor would yield an AUC of 0.0. To assess the reliability of the results obtained, all experiments were repeated in triplicates for alternative random seeds used for each experiment. We report mean performance across these prediction experiments and variability as plus and minus one standard deviation estimates.

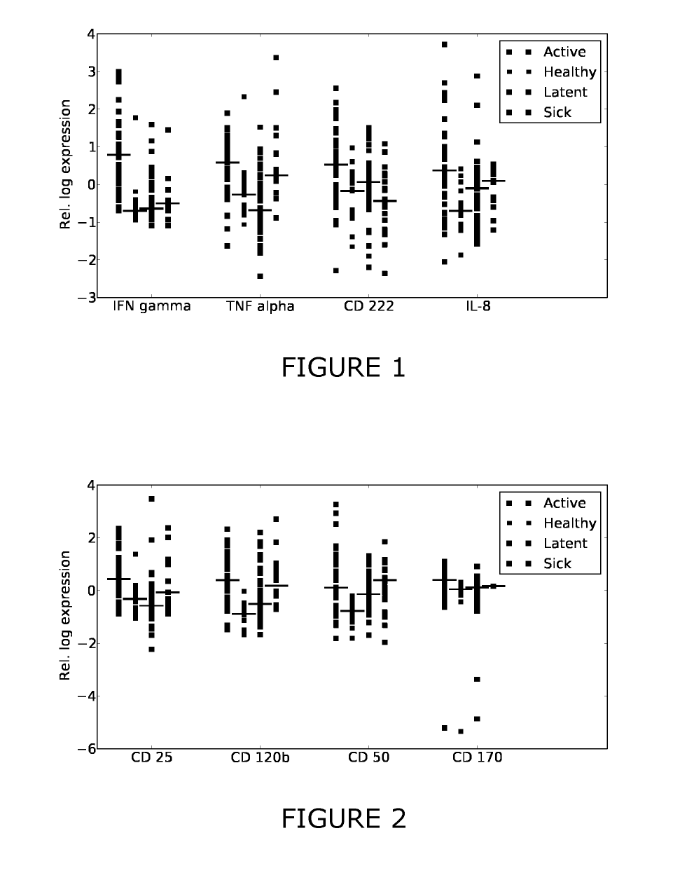

FIG. 1 shows scatter plots of IFN-gamma and TNF-alpha for the considered sample classes (healthy, active, latent, sick). Firstly, it can be seen that IFN-gamma and TNF-alpha yield complementary signatures when analyzed qualitatively. Whereas IFN-gamma appears to be most discriminative for active disease samples, TNF-alpha separates healthy samples and latent disease samples.

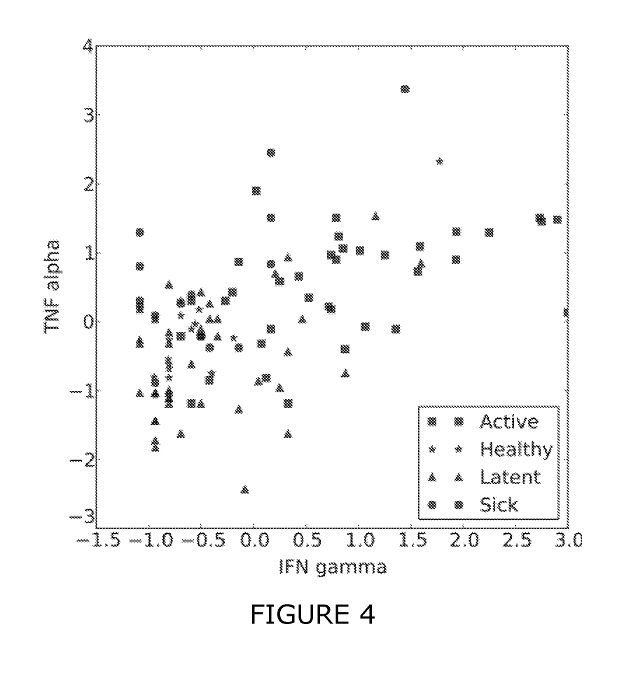

Combining TNF-alpha and IFN-gamma measurements for the same samples yields a compelling 2D-map of the different disease groups (FIG. 4), which is more discriminative than either antigen alone. Secondly, computational predictions were carried out using either individual antigens or their combination.

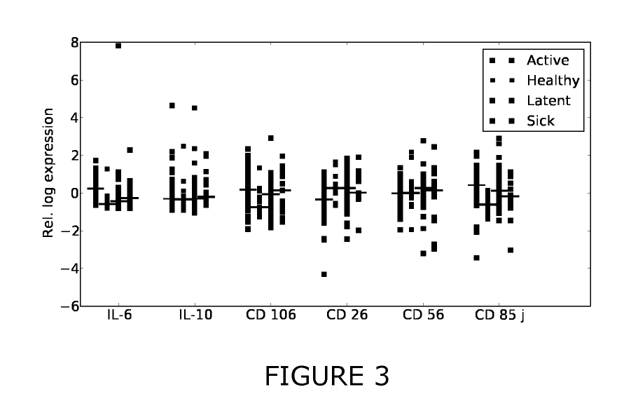

Table 2 shows the predictive performance for these alternatives and for different prediction tasks. The combination of both antigens consistently outperforms the single-antigen based approach. This quantitative assessment of the predictive abilities of different antigens is in line with the qualitative assessment gained from scatter plots (FIGS. 1-3).

TABLE 2
Predictive performance of different combinations of antigens
for alternative classification tasks

| | Healthy/ | Healthy/ | Healthy/ | Active/ | Sick/ | Sick/ | Sick/ |
|---|---|---|---|---|---|---|---|
| Antigen | TB | Active | Latent | Latent | TB | Active | Latent |
| IFN- | 0.47 | 0.87 | 0.35 | 0.84 | 0.49 | 0.78 | 0.39 |
| gamma | (0.02) | (0.01) | (0.03) | (0.01) | (0.02) | (0.02) | (0.05) |
| TNF | 0.43 | 0.69 | 0.30 | 0.77 | 0.43 | 0.50 | 0.59 |
| alpha | (0.03) | (0.01) | (0.07) | (0.01) | (0.04) | (0.07) | (0.03) |
| IFN- | 0.66 | 0.88 | 0.66 | 0.86 | 0.75 | 0.83 | 0.67 |
| gamma | (0.03) | (0.02) | (0.02) | (0.00) | (0.04) | (0.03) | (0.03) |
| & TNF- | | | | | | | |
| alpha | | | | | | | |
| Random | 0.58 | 0.71 | 0.64 | 0.52 | 0.68 | 0.67 | 0.66 |

The results in Table 2 show the area under the curve receiver operating characteristics (AUC). Error bars of plus and minus one standard deviation are given in brackets and have been estimated from three repeat experiments. The performance of a random predictor is deduced from the relative sizes of the two sample classes. The best performing antigen combination in any category is highlighted in bold. If several combinations perform equally well, they were all marked.

The results shown in Table 2 demonstrate that the combination of IFN-gamma and TNF-alpha is consistently a better predictor than any antigen in isolation.

### Example 2

### Effectiveness of Additional Biomarkers Combined with IFN-Gamma and TNF-Alpha

Additional antigens were identified which may complement the discriminative patterns observed from IFN-gamma and TNF-alpha. Firstly, FIGS. 1-3 show scatter plots for all antigens that were significantly differentially expressed (pv=0.05) between at least two of the considered sample classes (healthy, active, latent, sick). Several sCDs appear to add further axes of differentiation between the sample classes, complementing IFN-gamma and TNF-alpha. Table 3 summarizes the predictive performance when combining these CDs with TNF-alpha and IFN-gamma. This “joint” predictor performed at least as good as IFN-gamma and TNF-alpha and generally improved upon the results obtained with the two-antigen model for the prediction tasks healthy/TB and healthy/latent.

TABLE 3
Predictive performance of different combinations of antigens
for alternative classification tasks

| | Healthy/ | Healthy/ | Healthy/ | Active/ | Sick/ | Sick/ | Sick/ |
|---|---|---|---|---|---|---|---|
| Antigen | TB | Active | Latent | Latent | TB | Active | Latent |
| IFN- | 0.66 | 0.88 | 0.66 | 0.86 | 0.75 | 0.83 | 0.67 |
| gamma | (0.03) | (0.02) | (0.02) | (0.00) | (0.04) | (0.03) | (0.03) |
| & TNF- | | | | | | | |
| alpha | | | | | | | |
| Joint | 0.91 | 0.91 | 0.82 | 0.87 | 0.71 | 0.82 | 0.66 |
| | (0.03) | (0.03) | (0.00) | (0.04) | (0.03) | (0.03) | (0.04) |
| Random | 0.58 | 0.71 | 0.64 | 0.52 | 0.68 | 0.67 | 0.66 |

Table 3 shows the area under the curve receiver operating characteristics (AUC). Error bars of plus and minus one standard deviation are shown in brackets and have been estimated from three repeat experiments. The performance of a random predictor is deduced from the relative sizes of the two sample classes. The best performing antigen combination in any category is marked in bold. If several combinations perform equally well, they are all marked.

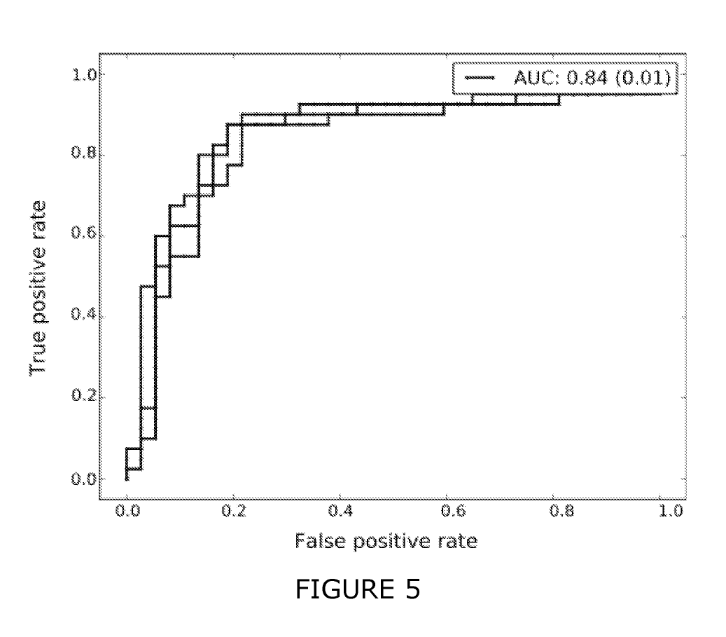

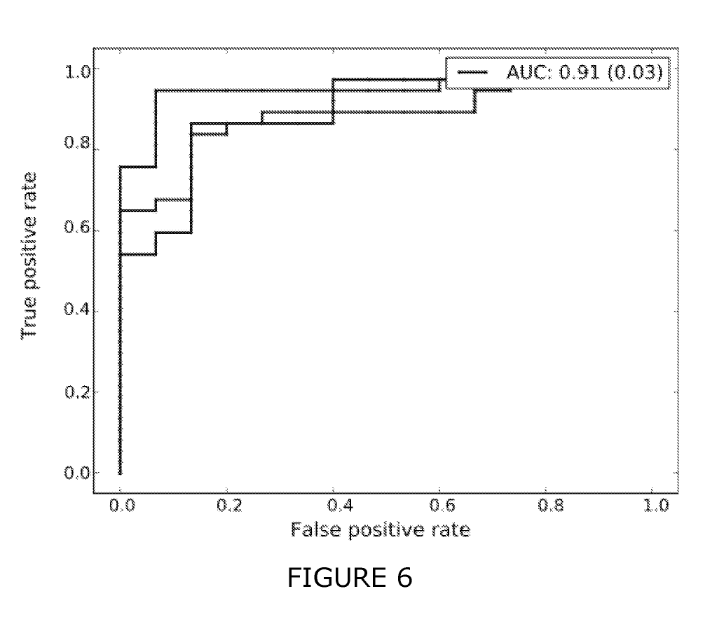

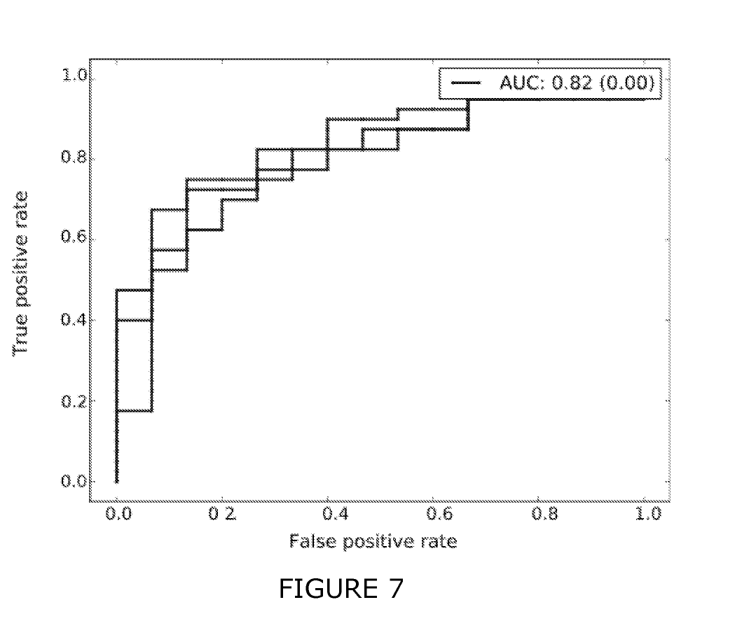

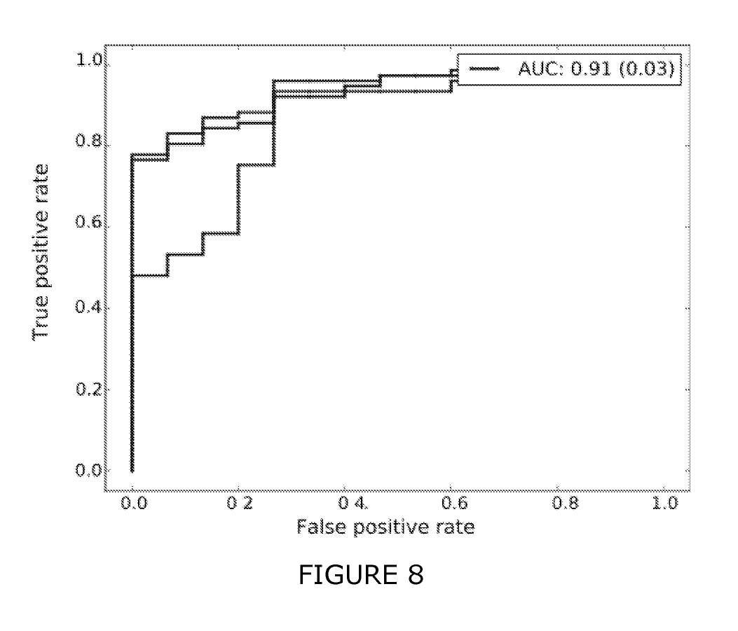

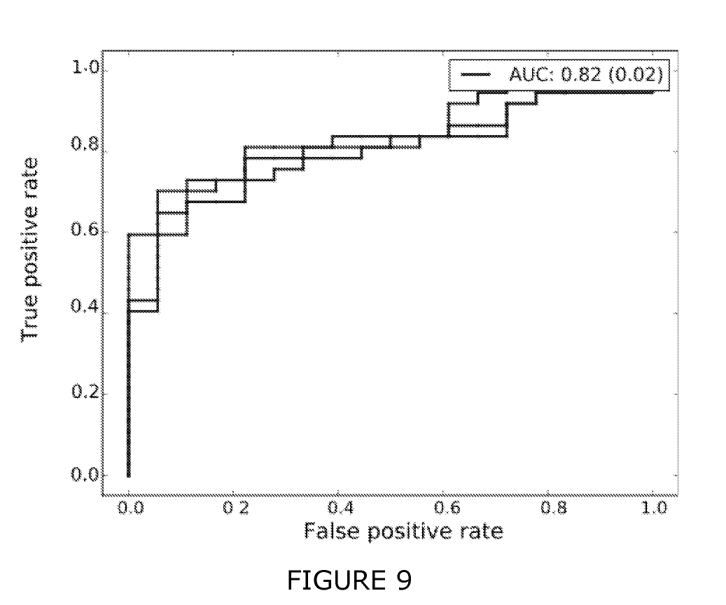

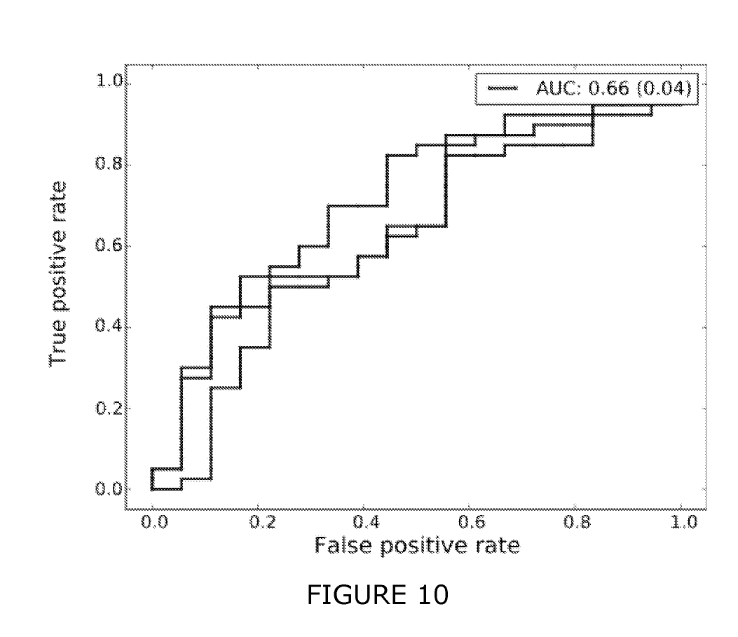

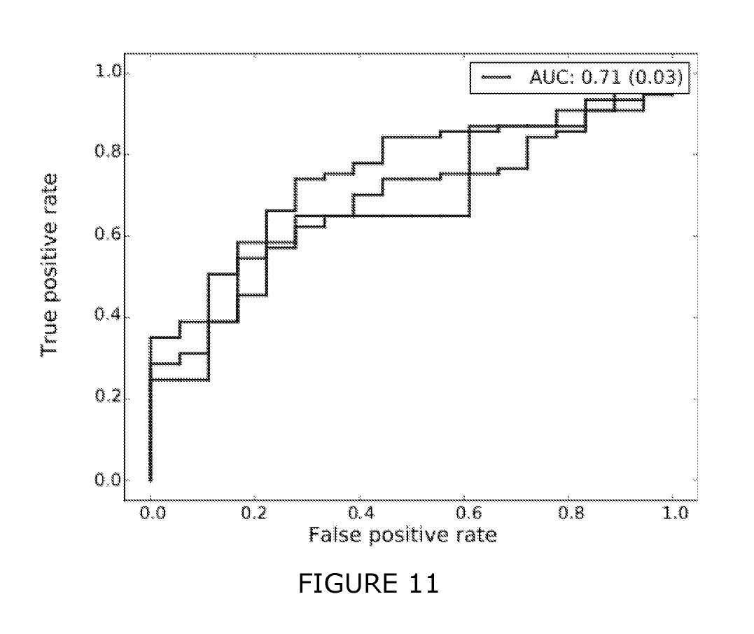

Finally, FIGS. 5-11 depict the receiver operating curves for the joint predictor and all 6 considered prediction tasks.

### Example 3

### Repeat of Active/Latent Model Using an Increased Patient Sample Size

The purpose of this experiment was to conduct a validation of the active/latent predictive signature identified in the initial screen which provided the results described in Example 1 and Example 2. To this end, unrelated samples were sourced from Imperial College London. These included the following numbers of active and latent TB cases:
Active TB: 94 samples (51: IGRA positive)
Latent TB: 89 samples (45: IGRA positive)

TABLE 4
Demographic and Patient Data for Each Sample Analysed

| | | | | | Diagnostic | |
|---|---|---|---|---|---|---|
| Sex | Age | BCG? | IGRA | Diagnosis | category | Group |
| F | 56 | Y | QFT+ | Pulmonary+ | 2 | ATB |
| | | | | Uveitis TB | | |
| F | 51 | N | QFT− | LTBI | 4B | LTBI |
| F | 21 | Y | NT | Lymph node TB | 1 | ATB |
| M | 21 | Y | QFT− | LTBI | 4A | LTBI |
| F | 49 | Y | QFT+ | Skin TB | 2 | ATB |
| F | 33 | Y | QFT+ | LTBI | 4B | LTBI |
| F | 25 | N | QFT−, | Pulmonary TB | 2 | ATB |
| | | | Tspot− | | | |
| F | 34 | Y | QFT+ | LTBI | 4B | LTBI |
| n/a | n/a | | QFT+ | n/a | 4B | ATB* |
| F | 55 | N | QFT− | LTBI | 4C | LTBI |
| F | 21 | Y | NT | Peritoneal TB | 1 | ATB |
| F | 39 | N | QFT+ | LTBI | 4B | LTBI |
| M | 34 | n/a | QFN+ | Pulmonary TB | 2 | ATB |
| F | 28 | N | NT | LTBI | 4B | LTBI |
| M | 35 | Y | QFT+, | Gastro Intestinal | 2 | ATB |
| | | | Tspot+ | TB | | |
| F | 56 | Y | Tspot+ | LTBI | 4B | LTBI |
| M | 34 | Y | NT | Pulmonary TB | 1 | ATB |
| M | 57 | Y | Tspot+ | LTBI | 4A | LTBI |
| F | 39 | Y | QFT+ | Lymph node TB | 2 | ATB |
| M | 38 | Y | Tspot+ | LTBI | 4C | LTBI |
| M | 48 | Y | NT | Spinal | 1 | ATB |
| | | | | (Bone/Joint)TB | | |
| F | 33 | N | QFN+ | LTBI | 4B | LTBI |
| F | 28 | N | QFT+ | Lymph node TB | 2 | ATB |
| F | 38 | Y | QFN+ X2 | LTBI | 4C | LTBI |
| M | 18 | N | QFN− IND | Pulmonary TB | 1 | ATB |
| M | 24 | Y | QFT+ | LTBI | 4B | LTBI |
| M | 23 | Y | QFN+ | Pulmonary TB | 1 | ATB |
| n/a | n/a | | | LTBI? | | LTBI* |
| F | 27 | Y | Tspot+ | Pulmonary TB | 2 | ATB |
| F | 34 | Y | Tspot+ | LTBI | 4B | LTBI |
| F | 68 | n/a | Tspot+ | Lymph node TB | 2 | ATB |
| M | 25 | N | QFT+ | LTBI | 4B | LTBI |
| F | 79 | N | Tspot+ | Skin TB | 2 | ATB |
| M | 30 | Y | Tspot+ | LTBI | 4C | LTBI |
| F | 31 | Y | QFT+ | TB Uveitis | 2 | ATB |
| n/a | n/a | n/a | sample | LTBI | not used | LTBI* |
| M | 27 | n/a | NT | Pulmonary TB | 1 | ATB |
| n/a | n/a | n/a | n/a | LTBI | 4C | LTBI* |
| F | 41 | Y | NT | Genito-urinary | 1 | ATB |
| | | | | TB | | |
| F | 78 | N | QFN+ | LTBI | 4B | LTBI |
| F | 28 | Y | NT | Lymph node TB | 1 | ATB |
| M | 44 | Y | QFN+ | LTBI | 4B | LTBI |
| M | 44 | n/a | NT | Abdominal + | 2 | ATB |
| | | | | Cervical LN TB | | |
| F | 40 | Y | QFN+ | LTBI | 4B | LTBI |
| M | 68 | n/a | NT | Pulmonary TB | 1 | ATB |
| M | 51 | Y | QFN− | LTBI | 4B | LTBI |
| | | | Tspot+ | | | |
| F | 58 | Y | Tspot+ | Lymph node/ | 2 | ATB |
| | | | | Uveitis TB | | |
| F | 31 | Y | QFN− | LTBI | 4B | LTBI |
| | | | Tspot+ | | | |
| M | 34 | Y | NT | TB Uveitis | 2 | ATB |
| M | 37 | Y | Tspot+ | LTBI | 4C | LTBI |
| F | 20 | Y | Tspot+ | Pulmonary TB | 1 | ATB |
| M | 34 | Y | QFN/Tspot+ | LTBI | 4A | LTBI |
| F | 20 | N | NT | Pulmonary TB | 1 | ATB |
| M | 41 | Y | QFN/Tspot+ | LTBI | 4C | LTBI |
| M | 40 | Y | Tspot+ | Pulmonary TB | 1 | ATB |
| F | 32 | Y | QFN+ | LTBI | 4B | LTBI |
| F | 44 | Y | QFT+ | Lymph node TB | 2 | ATB |
| F | 25 | Y | QFN+ | LTBI | 4B | LTBI |
| M | 31 | Y | NT | Retropharyngeal | 1 | ATB |
| | | | | and spinal TB | | |
| F | 44 | Y | QFN− | LTBI | 4B | LTBI |
| | | | Tspot+ | | | |
| n/a | n/a | | | | 4D | ATB |
| F | 31 | Y | QFN/Tspot+ | LTBI | 4C | LTBI |
| n/a | n/a | | | | | ATB* |
| F | 23 | n/a | QFN−ve | LTBI | 4B | LTBI |
| | | | Tspot+ | | | |
| M | 33 | Y | QFT+ | Lymph node TB | 2 | ATB |
| M | 29 | N | QFN+ | LTBI | 4B | LTBI |
| F | 38 | N | QFN+ | Pulmonary TB | 1 | ATB |
| M | 43 | N | QFN+ | LTBI | 4B | LTBI |
| M | 35 | Y | NT | Genito-urinary | 2 | ATB |
| | | | | TB | | |
| F | 30 | N | NT | LTBI | 4B | LTBI |
| F | 58 | Y | Tspot+ | Lymph node TB | 1 | ATB |
| F | 43 | Y | NK | LTBI | 4B | LTBI |
| M | 29 | Y | QFN+ | Lymph node TB | 1 | ATB |
| F | 62 | n/a | QFN+ | LTBI | 4B | LTBI |
| F | 53 | Y | NT | Lymph node TB | 1 | ATB |
| F | 33 | Y | QFN+ | LTBI | 4C | LTBI |
| F | 34 | Y | QFN+ | Lymph node TB | 1 | ATB |
| M | 46 | Y | QFN+ | LTBI | 4B | LTBI |
| F | 18 | N | QFN+ | Lymph node TB | 1 | ATB |
| | | | Tspot+ | | | |
| F | 33 | Y | QFN+ | LTBI | 4A | LTBI |
| M | 24 | Y | QFN+ | Pulmonary + LN | 1 | ATB |
| | | | | TB | | |
| M | 53 | Y | QFN+ | LTBI | 4B | LTBI |
| F | 21 | Y | QFN+ | Lymph node TB | 2 | ATB |
| F | 60 | Y | QFN+ | LTBI | 4B | LTBI |
| M | 19 | Y | QFN+ | Pulmonary/ | 1 | ATB |
| | | | | Spinal TB | | |
| F | 73 | Y | QFN+ | LTBI | 4A | LTBI |
| F | 29 | Y | QFN+ | Pulmonary TB | 2 | ATB |
| M | 32 | Y | QFN+ | LTBI | 4B | LTBI |
| M | 36 | n/a | NT | Left Neck Lump | 1 | ATB |
| | | | | TB | | |
| F | 27 | Y | QFN+ | LTBI | 4C | LTBI |
| M | 24 | n/a | NT | Lymph node TB | 1 | ATB |
| M | 55 | Y | QFN+ | LTBI | 4B | LTBI |
| F | 20 | n/a | Tspot+ | Pulmonary TB | 1 | ATB |
| M | 30 | Y | Tspot− | LTBI | 4A | LTBI |
| | | | QFN− | | | |
| F | 33 | Y | NT | Pulmonary TB | 1 | ATB |
| M | 59 | Y | Tspot+ | LTBI | 4B | LTBI |
| F | 59 | Y | QFN+ | Lymph node TB | 2 | ATB |
| F | 49 | Y | QFN+ | LTBI | 4B | LTBI |
| M | 52 | N | QFN+ | Lymph node TB | 2 | ATB |
| M | 54 | Y | Tspot+ | LTBI | 4C | LTBI |
| M | 41 | Y | NT | Pulmonary TB | 1 | ATB |
| M | 59 | n/a | Tspot+ | LTBI | 4C | LTBI |
| F | 41 | Y | QFN/Tspot− | Rt. Elbow and | 1 | ATB |
| | | | | axillary LN TB | | |
| M | 45 | N | QFN+ | LTBI | 4B | LTBI |
| M | 33 | n/a | QFN+ | MLN/Pulmonary | 1 | ATB |
| | | | Tspot+ | | | |
| M | 32 | Y | QFN+ | LTBI | 4C | LTBI |
| M | 30 | Y | NT | Pulmonary TB | 1 | ATB |
| M | 53 | Y | Tspot+ | LTBI | 4C | LTBI |
| M | 21 | Y | Tspot+ | Pulmonary TB | 1 | ATB |
| M | 42 | Y | QFN+ | LTBI | 4B | LTBI |
| | | | Tspot+ | | | |
| n/a | n/a | | | | | ATB* |
| M | 37 | Y | QFN+ | LTBI | 4B | LTBI |
| F | 29 | Y | NT | Pulmonary TB | 2 | ATB |
| M | 60 | n/a | QFN+ | LTBI | 4B | LTBI |
| n/a | n/a | n/a | QFN+ | n/a | 4D | ATB* |
| F | 32 | Y | QFN+ | LTBI | 4B | LTBI |
| M | 54 | Y | QFN+ | Pulmonary TB | 1 | ATB |
| M | 25 | N | QFN+ | LTBI | 4B | LTBI |
| F | 45 | Y | QFN+ | Lymph node TB | 1 | ATB |
| M | 36 | N | QFN+ | LTBI | 4B | LTBI |
| | | | Tspot+ | | | |
| F | 31 | Y | NT | Lymph node TB | 2 | ATB |
| F | 29 | N | QFN+ | LTBI | 4C | LTBI |
| M | 54 | n/a | QFN+ | MLN/Pulmonary | 1 | ATB |
| F | 32 | Y | QFN+ | LTBI | 4C | LTBI |
| | | | Tspot+ | | | |
| M | 22 | n/a | T spot+ | L. N TB (Rt. | 1 | ATB |
| | | | | submandibular) | | |
| M | 41 | N | QFN+ | LTBI | 4B | LTBI |
| M | 23 | n/a | QFN+ | Lymph node TB | 1 | ATB |
| F | 77 | Y | QFN+ | LTBI | 4B | LTBI |
| F | 76 | N | QFN+ | LN (Left Neck) | 2 | ATB |
| F | 48 | Y | T spot+ | LTBI | 4B | LTBI |
| F | 59 | Y | QFN+ | Lymph node TB | 2 | ATB |
| | | | | (Rt. Axilla) | | |
| M | 63 | N | T spot+ | LTBI | 4B | LTBI |
| F | 51 | n/a | T spot+ | Lymph node TB | 1 | ATB |
| F | 48 | N | QFN+ | LTBI | 4B | LTBI |
| F | 27 | Y | QFN+ | Pulmonary | 1 | ATB |
| F | 35 | N | QFN+ | LTBI | 4B | LTBI |
| F | 50 | Y | QFN+ | Pulmonary | 1 | ATB |
| F | 31 | N | QFN+ | LTBI | 4B | LTBI |
| M | 30 | N | QFN+ | Pleural/LN | 2 | ATB |
| | | | | Abdominal | | |
| M | 31 | Y | Tspot+ | LTBI | 4A | LTBI |
| M | 42 | Y | T spot+ | Skin TB | 2 | ATB |
| F | 32 | N | QFN+ | LTBI | 4B | LTBI |
| F | 33 | N | T spot+ | Pulmonary/ | 2 | ATB |
| | | | | Peritoneal TB | | |
| M | 61 | Y | T spot+ | LTBI | 4B | LTBI |
| M | 42 | Y | T spot+ | Sternal TB | 2 | ATB |
| M | 33 | Y | NK | LTBI | 4B | LTBI |
| F | 26 | n/a | QFN+ | Lymph node TB | 1 | ATB |
| M | 83 | n/a | T spot+ | LTBI | 4C | LTBI |
| M | 22 | N | TSPOT+ | Pulmonary | 1 | ATB |
| M | 24 | Y | T-spot + | LTBI | 4C | LTBI |
| F | 41 | Y | TSPOT+ | Uveitis/LN TB | 1 | ATB |
| M | 37 | Y | QFN+ | LTBI | 4B | LTBI |
| M | 27 | Y | TSPOT+ | Miliary/Pulmonary | 1 | ATB |
| M | 64 | N | QFN+ | LTBI | 4B | LTBI |
| M | 68 | Y | T spot + | MLN/Pulmonary | 1 | ATB |
| M | 25 | N | NT | LTBI | 4B | LTBI |
| M | 43 | Y | T spot + | LN (Left Neck) | 2 | ATB |
| M | 44 | Y | QFN+ | LTBI | 4B | LTBI |
| F | 54 | Y | T spot+ | Pan-Uveitis | 2 | ATB |
| M | 45 | Y | TSPOT+ | LTBI | 4B | LTBI |
| M | 40 | Y | T spot+ | Right neck gland | 1 | ATB |
| M | 24 | N | NT | LTBI | 4B | LTBI |
| M | 40 | n/a | T spot+ | Left axillary | 1 | ATB |
| | | | | abscess | | |
| M | 28 | Y | TSPOT −ve | LTBI | 4B | LTBI |
| M | 87 | Y | T spot+ | Pulmonary (M. Bovis) | 1 | ATB* |
| F | 18 | Y | NT | LTBI | 4B | LTBI |
| n/a | n/a | | T spot−ve | | 4D | ATB* |
| M | 32 | N | NT | LTBI | 4B | LTBI |
| M | 24 | N | T spot+ | Lymph node TB | 1 | ATB |
| F | 29 | Y | QFN+ | LTBI | 4B | LTBI |
| F | 39 | Y | NT | Pulmonary TB | 1 | ATB |
| M | 54 | n/a | QFN+ | LTBI | 4B | LTBI |
| F | 46 | Y | NT | Lymph node TB | 1 | ATB |
| M | 23 | Y | QFN+ | LTBI | 4B | LTBI |
| M | 54 | Y | NT | Pulmonary TB | 1 | ATB |
| F | 69 | Y | T | LTBI | 4B | LTBI |
| | | | spot+/QFN − | | | |
| M | 30 | Y | NT | Pulmonary TB | 1 | ATB |
| F | 47 | Y | QFN+ | LTBI | 4B | LTBI |
| M | 44 | Y | NT | Pulmonary+ | 2 | ATB |
| | | | | Uveitis TB | | |
| M | 24 | Y | T spot− | LTBI | 4C | LTBI |
| M | 33 | n/a | NT | Pulmonary TB | 1 | ATB |
| F | 47 | Y | T spot+ | LTBI | 4B | LTBI |
| M | 23 | Y | NT | Pulmonary TB | 1 | ATB |
| M | 51 | Y | QFT+ | LTBI | 4B | LTBI |
| M | 25 | n/a | NT | Cervical LN TB | 2 | ATB |
| M | 34 | N | QFT− | LTBI | 4B | LTBI |
| M | 68 | Y | NT | Pulmonary TB | 1 | ATB |
| F | 19 | Y | NT | LTBI | 4B | LTBI |
| F | 50 | Y | NT | Pulmonary + | 1 | ATB |
| | | | | Cervical LN TB | | |
| F | 49 | Y | QFT+ | LTBI | 4A | LTBI |
| M | 34 | N | NT | Lymph node TB | 2 | ATB |
| F | 27 | N | QFT+ | LTBI | 4B | LTBI |
| M | 29 | Y | NT | TB Uveitis | 2 | ATB |
| F | 44 | N | NT | LTBI | 4B | LTBI |
| M | 30 | N | NT | Pericarditis/Abdominal | 1 | ATB |
| | | | | TB | | |
| M | 55 | Y | QFT− | LTBI | 4B | LTBI |
| M | 18 | Y | QFT+ | Pulmonary TB | 2 | ATB |
| M | 33 | Y | QFT− | LTBI | 4B | LTBI |
| F | 20 | Y | NT | Pulmonary TB | 1 | ATB |

NB: The diagnostic category was calculated in accordance with Table 4 as described in Dosanjh et al (2008) Ann Int Med 148, 325-336.
ATB* refers to a sample previously labelled as TB but subsequently found not to be TB.
LTBI* refers to a sample previously labelled LTBI but subsequently found not to be LTBI.

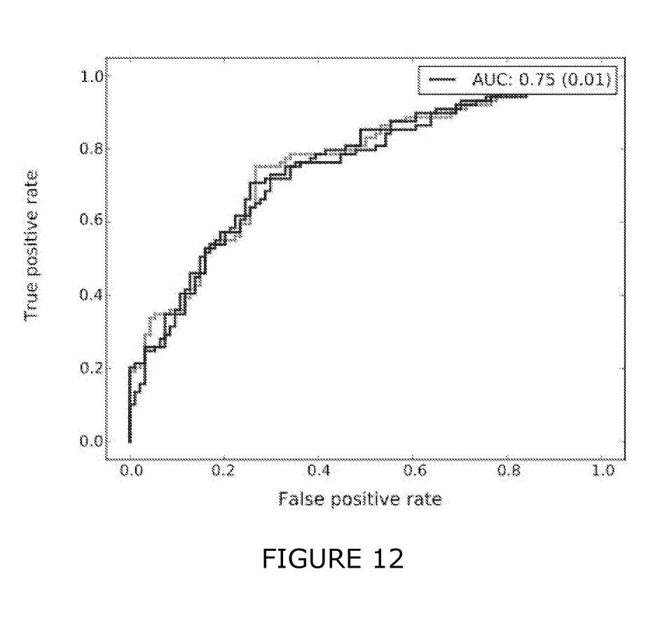

The results of this repeat study may be seen in FIG. 12. It was found that with the larger sample set, the discriminative joint signature to differentiate between active and latent TB could be validated in principle. However, the ROC performance dropped marginally from 0.84 (as previously shown in FIG. 5) to 0.75 (as shown in FIG. 12), however the overall ability to separate these two groups was confirmed on this substantially larger sample.

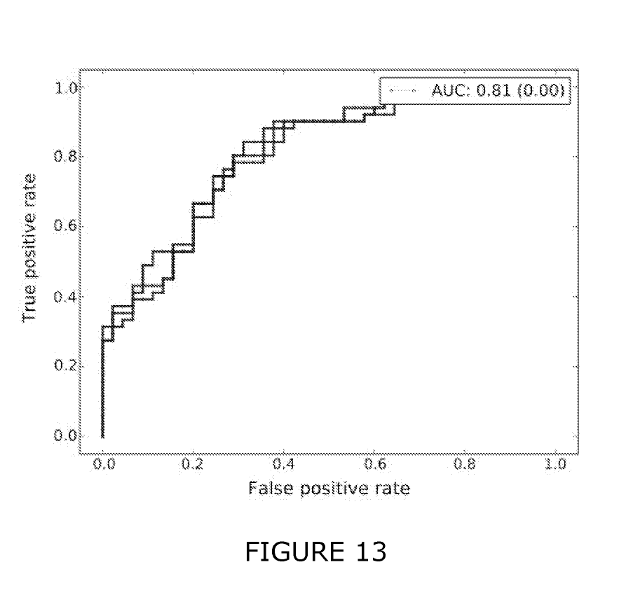

To demonstrate the merits of the proposed signature compared to the Interferon Gamma Release Assay (IGRA), the prediction experiment was repeated for the subset of samples which had a positive IGRA diagnosis (i.e. the 51 active samples and the 45 latent samples). FIG. 13 shows the corresponding ROC, which can be seen to have improved from 0.75 to 0.81 compared to using all samples. Therefore, the predictive accuracy increased compared to the result shown in FIG. 12, demonstrating that the antigen signature is both complementary and independent with respect to the IGRA status.

### Example 4

### Predictive Performance of Individual Antigens to Discriminate Active/Latent TB

The ability to differentiate between active and latent TB is mainly based on IFN-gamma and other biomarkers that capture these pathways. Table 4 below lists predictive ability for individual antigens based on the values obtained from (A) the patient samples listed in Table 4 and (B) the subset of patient samples within Table 4 which were marked as IGRA positive. The results are shown in Table 5 wherein the antigens marked in bold font are believed to be more predictive than chance.

TABLE 5
Predictive performance of individual antigens

| | | B |
|---|---|---|
| | A | IGRA Positive |
| Antigen | All Samples | Samples |
| sCD 25 | 0.69 | 0.76 |
| sCD 50 | 0.46 | 0.63 |
| sCD 120b | 0.66 | 0.73 |
| sCD 170 | 0.68 | 0.60 |
| sCD 222 | 0.49 | 0.42 |
| IFN-gamma | 0.64 | 0.70 |
| IL-1β | 0.51 | 0.34 |
| IL-6 | 0.50 | 0.39 |
| IL-8 | 0.48 | 0.39 |
| IL-10 | 0.49 | 0.38 |
| IL-12 | 0.49 | 0.41 |
| TNF alpha | 0.47 | 0.63 |

The results from Table 5 demonstrate the ability of sCD25, sCD120, sCD170 and IFN-gamma to discriminate between active and latent TB in both IGRA negative and positive and IGRA positive patient sample subsets.

### Example 5

### Effectiveness of IFN-Gamma and sCD170 as TB Biomarkers

In view of the results from Example 4, the use of IFN-gamma and sCD170 was assessed for their effectiveness to discriminate between active and latent TB.

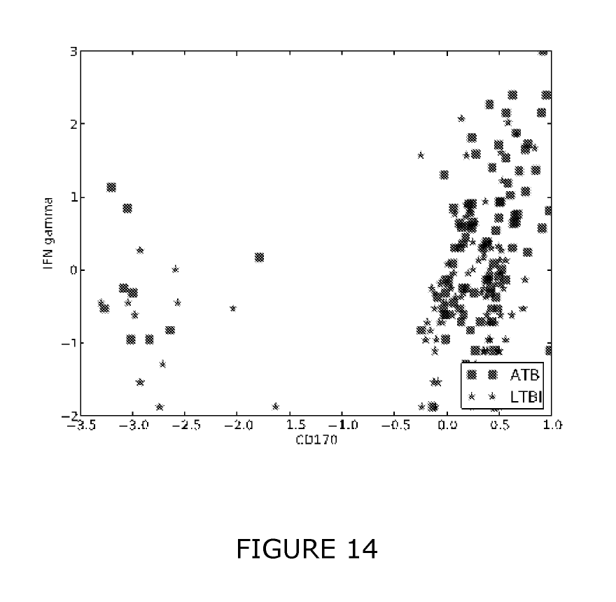

An analysis was conducted in an analogous manner to that described in Example 1 and Table 2 using the IGRA positive samples listed in Table 4. FIG. 14 shows a scatter plot of combined IFN-gamma and sCD170 measurements for the considered sample classes (active and latent TB). Firstly, these results provide a compelling 2D-map of the different disease groups (FIG. 14), which is more discriminative than either antigen alone. Secondly, computational predictions were carried out with the same samples using either individual antigens or their combination.

The results of this analysis are shown in Table 6 wherein it can be seen that the combination of IFN-gamma and sCD170 yielded better discriminative signals than either of these antigens alone.

TABLE 6
Predictive performance of different combinations of antigens
for alternative classification tasks

| Antigen | Active/Latent |
|---|---|
| IFN-gamma | 0.70 |
| sCD170 | 0.60 |
| IFN-gamma & sCD170 | 0.77 |

### Example 6

### Effectiveness of Additional Biomarkers Combined with IFN-Gamma and sCD170

Additional antigens were identified which may complement the discriminative patterns observed from IFN-gamma and sCD170. This analysis was conducted in an analogous manner to that described in Example 2 and Table 2 using the IGRA positive samples listed in Table 4. Table 7 summarizes the predictive performance when combining these CDs with sCD170 and IFN-gamma. This “joint” predictor performed at least as good as IFN-gamma and sCD170 and generally only marginally improved upon the results obtained with the two-antigen model for the prediction tasks demonstrating the effectiveness of the combination of IFN-gamma and sCD170.

TABLE 7
Predictive performance of different combinations of antigens
for alternative classification tasks

| Antigen | Active/Latent |
|---|---|
| IFN-gamma & sCD170 | 0.76 |
| Joint | 0.81 |

1. A method for diagnosing tuberculosis and/or monitoring tuberculosis in an individual comprising:
detecting analyte biomarker sCD170 in a biological sample from the individual, quantifying analyte biomarker sCD170 in the biological sample from the individual, or both;
optionally detecting analyte biomarker IFN gamma in the biological sample from the individual; and
optionally quantifying analyte biomarker IFN gamma in the biological sample from the individual.

2. The method of claim 1, wherein the method comprises:
detecting analyte biomarker sCD170, quantifying analyte biomarker sCD170, or both; and
detecting analyte biomarker IFN gamma, quantifying analyte biomarker IFN gamma, or both.

3. The method according to claim 1, wherein the diagnosing comprises differential diagnosis between any one of: active tuberculosis and latent tuberculosis; active tuberculosis and healthy control(s); latent tuberculosis and healthy control(s); active tuberculosis and sick control(s); and latent tuberculosis and sick control(s).

4. The method according to claim 1, wherein the diagnosing comprises differential diagnosis between active tuberculosis and latent tuberculosis.

5. The method according to claim 1, which additionally comprises quantifying or detecting one or more further analyte biomarkers selected from IL-1β, IL-6, IL-8, IL-10, IL-12p70, sCD4, sCD25, sCD26, sCD32b/c, sCD50, sCD56, sCD66a, sCD83, sCD85j, sCD95, sCD106, sCD120b, sCD121b, sCD127, sCD154, sCD222, sCD226, sCDw329 and TNF alpha.

6. The method according to claim 1, which additionally comprises quantifying or detecting one or more further analyte biomarkers selected from IL-6, IL-8, IL-10, sCD25, sCD26, sCD50, sCD56, sCD85j, sCD106, sCD120b, sCD222 and TNF alpha.

7. The method according to claim 1, which additionally comprises quantifying or detecting one or more further analyte biomarkers selected from IL-8, sCD25, sCD50, sCD120b, sCD222 and TNF alpha.

8. The method according to claim 1, which additionally comprises quantifying or detecting each of the following further analyte biomarkers: IL-8, sCD25, sCD50, sCD120b, sCD222 and TNF alpha.

9. The method according to claim 1, which additionally comprises quantifying or detecting each of the following further analyte biomarkers: IL-6, IL-8, IL-10, sCD25, sCD26, sCD50, sCD56, sCD85j, sCD106, sCD120b, sCD222 and TNF alpha.

10. A method of diagnosing tuberculosis in an individual, comprising:
(a) obtaining a biological sample from the individual;
(b) detecting and/or quantifying the amount of analyte biomarker sCD170, optionally in combination with analyte biomarker IFN-gamma and/or one or more additional analyte biomarkers of claim 5; and
(c) comparing the amounts of the analyte biomarkers in the biological sample with the amounts present in one or more control samples, such that a difference in the level of the analyte biomarkers in the biological sample is indicative of a diagnosis of tuberculosis in the individual.

11. The method according to claim 10, wherein a higher level of sCD170 in the biological sample compared with the control sample is indicative of a diagnosis of tuberculosis.

12. A method of monitoring efficacy of anti-microbial therapy in a subject having or suspected of having tuberculosis, comprising detecting and/or quantifying sCD170, optionally in combination with IFN-gamma and/or one or more additional analyte biomarkers of claim 5, in a sample from the subject.

13.-22. (canceled)

23. The method according to claim 10, wherein the detecting and/or quantifying is performed using an immunological method.

24. The method according to claim 10, wherein the detecting and/or quantifying is performed using a biosensor or a microanalytical, microengineered, microseparation or immunochromatography system.

25. The method according to claim 10, wherein the biological sample is whole blood, serum, plasma, non-activated serum, tissue fluid, cerebrospinal fluid (CSF), synovial fluid, follicular fluid, seminal fluid, amniotic fluid, milk, urine, pleural fluid, ascites, bronchoalveolar lavage, saliva, sputum, tears, perspiration, lymphatic fluid, aspirate, bone marrow aspirate and mucus, or an extract or purification therefrom, or dilution thereof.

26.-27. (canceled)

28. The method of treating tuberculosis in an individual in need thereof, wherein the method comprises:
(a) diagnosing tuberculosis in the individual according to the method of claim 10; followed by
(b) administering an anti-tuberculosis medicament to the individual in the event of a positive diagnosis for tuberculosis.

29. The method according to claim 28, wherein the anti-tuberculosis medicament is:
one or more first line medicaments selected from ethambutol, isoniazid, pyrazinamide and rifampicin; or
one or more second line medicaments selected from aminoglycosides, amikacin, kanamycin, polypeptides, capreomycin, viomycin, enviomycin, fluoroquinolones, ciprofloxacin, levofloxacin, moxifloxacin, thioamides, ethionamide, prothionamide, cycloserine and terizidone; or
one or more third line medicaments selected from rifabutin, macrolides, clarithromycin, linezolid, thioacetazone, thioridazine, arginine, vitamin D and R207910; or
combinations thereof.

30. The method according to claim 10, wherein the tuberculosis is latent tuberculosis or active tuberculosis.

31. The method according to claim 30, wherein the active tuberculosis is extrapulmonary tuberculosis.

32. A kit comprising a biosensor capable of detecting and/or quantifying analyte biomarker sCD170, optionally in combination with analyte biomarker IFN-gamma and/or one or more additional analyte biomarkers of claim 5, suitable for use in diagnosing and/or monitoring tuberculosis.

33. (canceled)
